(19) United States
(12) Patent Application Publication
(10) Pub. No.: US 2010/0161345 A1
(43) Pub. Date: Jun. 24, 2010

(54) MEDICAL DATA TRACKING, ANALYSIS AND AGGREGATION SYSTEM

(75) Inventors: Perry M. Cain, Akron, OH (US); Angelo Rago, Walnut Creek, CA (US); John M. Burkle, Cuyahoga Falls, OH (US); Nicholas A. Kuzmik, Akron, OH (US); David T. Espersen, Cuyahoga Falls, OH (US); Michael A. Markwood, Barberton, OH (US)

Correspondence Address: THOMPSON HINE L.L.P.;Intellectual Property Group, P.O. BOX 8801, DAYTON, OH, 45401-8801, US

(73) Assignee: INTEGRATED SURGICAL SOLUTIONS, LLC, Fairlawn, OH (US)

(21) Appl. No.: 12/342,855

(22) Filed: Dec. 23, 2008

(51) Int. Cl. G06Q 50/00; G06F 17/30

(52) U.S. Cl. 705/2; 705/28; 707/E17.044; 707/E17.014

(57) ABSTRACT

A medical item tracking method including storing inventory levels of items which are for use in medical procedures and receiving input for reserving the use of a facility for a medical procedure, which medical procedure is projected to use at least one of the inventoried items. The input identifies at least one individual projected to participate in the medical procedure. The method further includes retrieving data relating to the predicted usage of the inventoried item in the medical procedure, wherein the data is based upon the participation of the at least one individual. The method further includes adjusting the levels of inventory based upon the retrieved predicted usage data.

The present invention is directed to a tracking, analysis and data aggregation system, and more particularly, to a tracking, analysis and data aggregation system for use with medical supplies and associated items.

### BACKGROUND

Medical facilities, such as hospitals, physician's offices, outpatient facilities and the like generate high volumes of data and information which is required to be tracked for various purposes, including insurance, regulatory, accounting, billing, cost control, inventory management and the like. It may also be beneficial for such facilities to track information relating to the costs of particular procedures and medical items, costs generated by particular physicians, success/failure rates of medical items and procedures, trends relating to the usage and ordering of certain items and medical procedures, etc. Medical facilities may also be required to track and report information to various entities, such as the Food and Drug Administration (“FDA”), device manufacturers, suppliers or vendors, patients, doctors, insurance companies, etc. In some cases, such as when medical facilities utilize implanted devices, such medical facilities also need to track expiration dates and unique identifiers for each implant.

Thus various disparate forms of data are generated at various times, which data needs to be aggregated, tracked and processed. In addition, the data can correspond to a large number of items and events which have varying qualities that are to be tracked. Moreover, relatively high volumes of data may be generated at rapid rates as a high number of medical procedures may be scheduled and carried out at a particular facility in a given day.

In addition, the staff at such medical facilities may be required to serve in multiple roles, which increases the complexity of proper data tracking and reporting. Finally, existing medical facilities may utilize various types of inventory and tracking software that are not necessarily compatible. Accordingly, it can be seen that such medical facilities have multiple types of data, in various formats, entered at various times and locations by various personnel, and supported on different platforms, leading to difficulties in data management and aggregation.

### SUMMARY

In one embodiment, the present invention is a system and method in which disparate data utilized and created by a medical facility (and elsewhere), in its various forms and locations, is collected in small datasets at different times and by different actions, and then integrated into a single system/method such that the data can be processed and managed to provide multiple outputs in various forms. More particularly in one certain embodiment, the invention is a medical item tracking method including storing inventory levels of items which are for use in medical procedures and receiving input for reserving the use of a facility for a medical procedure, which medical procedure is projected to use at least one of the inventoried items. The input identifies at least one individual projected to participate in the medical procedure. The method may further include retrieving data relating to the predicted usage of the inventoried item in the medical procedure, wherein the data is based upon the participation of the at least one individual. The method further includes adjusting the levels of inventory based upon the retrieved predicted usage data.

### BRIEF DESCRIPTION OF THE DRAWINGS

FIG. 1 is a schematic representation of a relation between a plurality of users, a host and a supplier as implemented in one embodiment of the system and method disclosed herein;

FIG. 2 is a screen shot showing inventory levels of certain items for a particular user;

FIG. 3 is a screen shot showing historical inventory information for a particular item;

FIG. 4 is a screen shot showing a new product entry form;

FIG. 5 is a screen shot showing various details for an item in inventory;

FIG. 6 is a screen shot showing various options with respect to the placing of an order;

FIG. 7 is a screen shot showing bundling options for a particular order;

FIG. 8 is a screen shot showing certification options for various items;

FIG. 9 is a screen shot showing supply list label management options;

FIG. 10 is a screen shot showing information relating to received orders;

FIG. 11 is a screen shot showing information relating to usage parameters for a particular item;

FIG. 12 is a screen shot showing information relating to an accounting tracking designation for a particular item;

FIG. 13 is a screen shot showing inventory reconciliation information for a particular item;

FIG. 14 is a screen shot showing information relating to a return authorization form;

FIG. 15 is a flow chart illustrating the various steps, with annotations, a user may follow to return an item under one embodiment of the system and method disclosed herein;

FIG. 16 is a screen shot showing a preference data form;

FIG. 17 is a screen shot showing predictive costs for various procedures based upon their associated preference data forms;

FIG. 18 is an example of a surgical log;

FIG. 19 is a screen shot showing the entry of a preference card exception;

FIG. 20 is a screen shot showing an actual expense report for a procedure;

FIG. 21 is a screen shot showing a scheduling calendar;

FIG. 22 is a screen shot showing a search function for the scheduling calendar;

FIG. 23 is a flow chart, with various annotating comments, illustrating how a user is integrated into one embodiment of the system and method described herein;

FIG. 24 is a flow chart, with various annotating comments, illustrating various steps involving the use, tracking, and reordering of items under one embodiment of the current system and method;

FIG. 25 is a flow chart showing the functionality of a personal electronic device (mobile device);

FIG. 26 is a flow chart illustrating implementation and operation of the system and method, with further particular details relating to supplier and user relationships;

FIG. 27 is a flow chart showing various features of one embodiment of the system and method disclosed herein, particularly with respect to child and parent facilities; and

FIG. 28 is a schematic representation showing certain aspects of the system and method.

It should be noted that the screenshots provided herein demonstrate only certain information utilized by one embodiment of the present system and method, as well as only one possible format for presenting and gathering information.

### DETAILED DESCRIPTION

1. Definitions and Background

Prior to describing the present system and method in greater detail, certain terms used herein will first be defined. “Computer” means computers, computer components and elements of a computer, such as hardware, firmware, virtualized hardware and firmware, a combination thereof, or software in execution. One or more computers can reside in or on a server in various embodiments and the server can itself be comprised of multiple computers. One or more computers can reside within a process and/or thread of execution and a computer can be localized at one location and/or distributed between two or more locations.

“Personal electronic device” means a handheld, stationary (i.e. placeable on a table-top) or wearable electronic device which can receive digitized inputs and/or provide digitized outputs, such as an electronic organizer, personal digital assistant (“PDA”), mobile phone, or the like. The personal electronic device may have the capability to scan and recognize bar codes, radio frequency identification (“RFID”) tags, and/or other identifications, receive manually entered information (i.e. via by a keypad, digitizer, or keyboard), receive information through an audio input and extract meaningful information therefrom (i.e. utilizing voice recognition technology), receive information via optical character recognition technology, menu-driven (hierarchical) or icon-based inputs, or utilize various other means to receive, record and/or transmit information.

“Computer communications” means communication between two or more computers and/or personal electronic devices, and can take the form of, for example, a network transfer, a file transfer, an applet transfer, an email, a hypertext transfer protocol (“HTTP”) message, a datagram, an object transfer, a binary large object (“BLOB”) transfer, and so on. Computer communication can occur across a variety of mediums by a variety of protocols, for example, a wireless system (e.g., IEEE 802.11), an Ethernet system (e.g., IEEE 802.3), a token ring system (e.g., IEEE 802.5), a local area network (“LAN”), a wide area network (“WAN”), BLUETOOTH® communications, a point-to-point system, a circuit switching system, a packet switching system, wireless or satellite communication systems, and various other systems.

“Software” means one or more computer readable and/or executable instructions that cause a computer, personal electronic device or other electronic device to perform functions, actions and/or behave in a desired manner. The instructions may be embodied in various forms such as routines, algorithms, modules, methods, threads, and/or programs. Software may also be implemented in a variety of executable and/or loadable forms including, but not limited to, stand-alone programs, function calls (local and/or remote), servelets, applets, instructions stored in a memory, part of an operating system or browser, bytecode, interpreted scripts and the like. It should be appreciated that the computer readable and/or executable instructions can be located in one computer or the like and/or distributed between two or more communicating, co-operating, and/or parallel processing computers or the like and thus can be loaded and/or executed in serial, parallel, massively parallel and other manners. It should also be appreciated that the form of software may be dependent on various factors, such as the requirements of a desired application, the environment in which it runs, and/or the desires of a particular designer/programmer.

“Webpage” means any document written or encoded in a mark-up language, or dynamically created by software including, but not limited to, hypertext mark-up language (“HTML”), virtual reality modeling language (“VRML”), dynamic HTML, extended mark-up language (“XML”) or related computer languages and scripts, including scripts and other resources contained with a mark-up language shell such as FLASH® or JAVASCRIPT® applets, as well as any collection of documents reachable through one specific Internet address or at one specific website, or any document obtainable through a particular uniform resource locator (“URL”). To “display a webpage” means to undertake actions necessary to render at least a portion of the information on the webpage available to the computer user. As such, the phrase includes, but is not limited to, the static visual display of static graphical information, audible production of audio information, animated visual display, and visual display of video stream data.

“Website” means at least one webpage, or a plurality of webpages, virtually linked to form a coherent group.

“Web browser” means any software program running on a computer which can display text, graphics, or both, from webpages or websites. Examples of commercially available web browsers include, without limitation, MICROSOFT® INTERNET EXPLORER®, MOZILLA® FIREFOX®, APPLE® SAFARI®, GOOGLE® CHROME™, OPERA™ browsers, and the like.

“Web server” means a computer, computers, or software configured to operate at least in part as a server capable of serving or providing information associated with at least one webpage to a web browser at the request of a user.

“Database” means any of a number of different data stores that provide searchable indices for storing, locating and retrieving data, including without limitation, relational databases, associative databases, hierarchical databases, object-oriented databases, network model databases, dictionaries, flat file/XML datastores, flat file systems with spidering or semantic indexing, and the like.

The system and method described herein, broadly speaking and in one embodiment, is a data tracking and aggregation system in which data relating to various components and events is tracked and processed, resulting in powerful data mining and aggregation, and incorporating the use of predictive algorithms and the presentation of processed information in a unique manner. The system and method may thus be used in a variety of environments in which it is desired to monitor items, components, events or the like, and track their use in and effect upon various procedures and processes. However, for the sake of providing a specific example in which particular details are described to illustrate certain features of the invention, the system and method described herein will often be described in the context of the acquisition, storage, use, tracking and reporting of medical events and items in various medical procedures.

More particularly, in the illustrative examples presented herein, the particular medical procedure discussed takes the form of ocular lens replacement surgery in which an intra-ocular lens (“IOL”) is used to replace the ocular lens of a patient that has developed cataracts or other problems. The illustrative system and method thus involves and tracks implants/IOLs and various ancillary medical items utilized in or associated with an implantation procedure. Such a procedure is often carried out by a physician, assisted by a staff, at an ambulatory surgical center (“ASC”). However, it should be noted that the example described herein is presented only for ease of explaining the functionality of the invention in a particular setting, and is not intended to limit the scope of the invention. In particular, it should be understood that the system and method disclosed herein can be used in a variety of other medical fields/procedures such as heart surgery, joint or orthotic replacement, breast implant, the handling of blood, tissue, bone, glaucoma stents, retinal or cochlear implants, stents, pacemakers, etc. In addition, the system and method need not necessarily be used in the medical field, and can be used in a variety of other settings, such as in veterinarian operations, manufacturing operations, business operations, handling, shipping and/or storage operations, assembly operations or in the fields of office supply, automotive repair, food service, computer repair, home repair, retail stores, etc., or nearly any setting in which items (particularly inventoried items) and/or events are desired to be tracked.

2. System Architecture

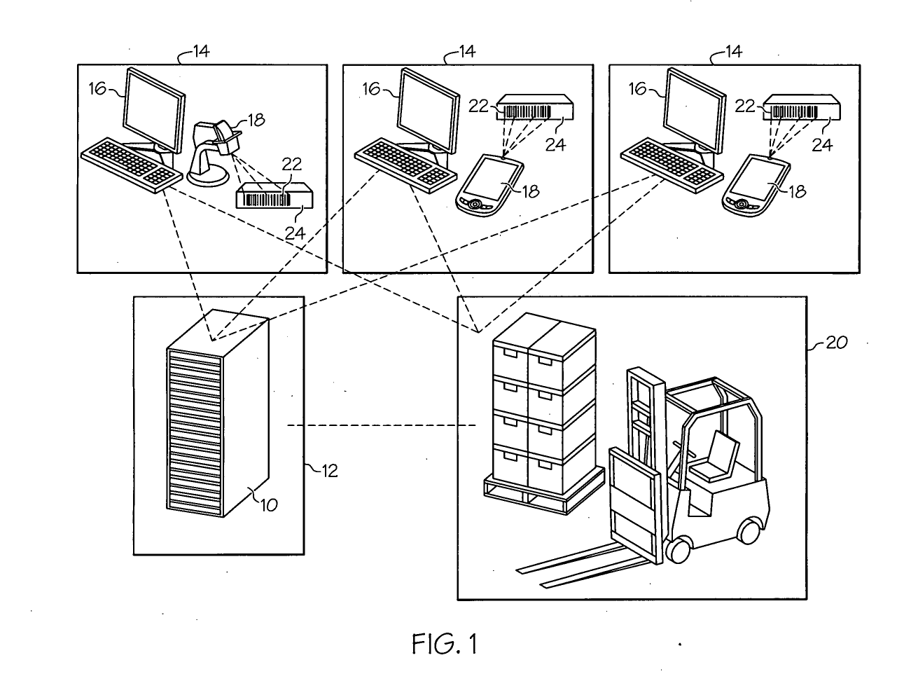

The system and method disclosed herein can be implemented and configured in a wide variety of manners. However, in one embodiment, the software for operating the system and method disclosed herein, as well as the associated databases used by the system and method, are stored on a central computer or system 10 (FIG. 1), which is operated by, controlled by, or accessible by an operator or host company/entity 12. The computer 10/host 12 is connected to a user 14 (or a plurality of users 14) and/or the user's computer 16 by computer communications or various other means, such as fax, email, an electronic data interchange (“EDI”) network, the Internet, point-to-point and networked communication links, or the like. In one case each “user” 14 is considered the entity which utilizes the software, system and method provided by the host 12 and/or stored on the host's computer 10. In the illustrative example outlined above, one or each user 14 may be an ASC or other medical services provider, which acts through its various employees, officers, owners, operators, directors, and agents.

The host 12 may provide a user interface by which various distributed users 14 can access the system, method and databases operated and maintained by the host 12. For example, in one embodiment, the user interface is made available via the Internet. In this case, a user 14 can access the software, methods and databases of the system and method by navigating to a particular webpage or website using the user's computer 16 and an appropriate web browser. The computer 10 may include or be connected to a web server to serve the webpage/website in response to the request from the user's computer 16.

After accessing the host's webpage/website, the user 14 may then be required to log in, such as by using a username and password, so that access is controlled and the system remains secure. The computer 10 and the user's computer 16 may communicate via a secured tunnel or interface such as a virtual private network (“VPN”) or secure sockets layer (“SSL”) to thwart third party interlopers from accessing or capturing information transferred between the web server and the user's 14 web browser. Various other security systems and/or secure log-in methods may be implemented as desired.

Once the user 14 has navigated to the appropriate webpage/website, and logged in, or otherwise accessed the host's software/computer 10, the user 14 may be provided access to the records, databases, methods, systems and preferences associated with that user 14. In this manner, a user 14 is not required to load any software on its computer 16, and is not required to store any data or information on its computer 16 or other storage devices (although cookies or other data or program fragments may be stored on the user's computer 16). Alternately, if desired, the user 14 may load software on its computer 16, or parts thereof, to operate the system and method, and/or to enable the user's computer 16 to be disconnected and not in communication with the host computer 10 while still allowing off-line entry of information to be processed at a later time. The user 14 may also use terminals (not shown) that are directly connected to the user's computer 10.

One or each user 14 may also utilize one or more personal electronic devices 18 to upload and download data and record events, as will be described in greater detail below. The personal electronic device 18 may store information relating to the entry of information, noting the time associated with each data entry. The personal electronic device 18 may then forward the information to the user's computer 16 when the personal electronic device 18 is coupled to the user's computer 16 (i.e. via a cable, docking system, hot-sync system, wireless connection, etc.)

Alternately the personal electronic device 18 may directly communicate with the host's software/computer 10. In this case, in addition to possibly storing information for later uploading, the personal electronic device 18 may be directly connected (i.e., by a wired or wireless connection) to the user's computer 16 (or the host computer 10) at the time the data is read/entered. Thus data may be automatically provided from the personal electronic device 18 in real-time or near-real time.

It should be understood that the personal electronic device 18, unless specifically noted otherwise, may have the same or similar capabilities as the user's computer 16. Thus a user 14 can utilize a personal electronic device 18 to connect to the host computer 10 (either directly or indirectly) and fully utilize the system and method described herein as if the user 14 were operating from its computer 16. In this manner the personal electronic device 18 allows a user to access, process, and modify data from a remote location. Of course, any of a wide variety of other data tracking and gathering means may be used with the user's computer 16 and/or personal electronic device 18 without departing from the scope of the present invention, including simply entering information directly into the user's computer 16 and/or host computer 10 by scanning and recognizing bar codes, and/or radio frequency identification (“RFID”) tags, and/or other identifiers, receiving manually entered information (i.e. via by a keypad, digitizer or keyboard), receiving information through an audio input (i.e. utilizing voice recognition technology), through optical character recognition technology, through a menu-driven (hierarchical) or icon-based inputs, through connections with externally provided data, such as may be provided by a vendor/supplier, receiving inputs from wireless or satellite systems, or by various other means.

The host 12 and/or user 14 may be connected to a supplier 20 by computer communications or other means, such as facsimile, email, EDI, internet or the like. The supplier 20 can be a vendor, manufacturer, warehouse, or the like (collectively termed a “supplier” herein) which can supply goods to the host 12 and/or user 14 for use by the user 14. In the example described herein the supplier 20 may be a manufacturer or vendor of implants and related medical supplies.

3. Inventory Management

a. Item/Product Identifiers

In one embodiment, the present system and method is configured to store and process information related to items/products, including uniquely identified medical items and non-uniquely identified medical items. For example, in one case, the uniquely-identified medical items take the form of an implant designed to be used in the human body, and more specifically, an IOL. In this case, FDA regulations require each implant to have its own unique identifier/unique identification information such that each unique implant is traceable to individual patients, which enables recalls to be quickly and efficiently instituted to allow the performance of the implants to be tracked, etc.

In the cases of some implants (i.e., orthopedic implants), the unique identifier may be marked directly on the implant. However, in the case of an IOL, glaucoma stent, heart stent, etc., it may not be practical or convenient to mark the item with its unique identifier. In this case, then, the box, packaging, labels, or other materials associated with the IOL may carry the unique identifier.

In many cases an item does not have a unique identifier, or it is not necessary to track unique identifiers. For example, in the case of an ASC that implants IOLs, various other medical supplies used in an ocular lens replacement procedure may be desired to be tracked, such as towels, scalpels, gloves, sutures, saline solution, gurneys, pharmaceuticals, etc. In this case the non-unique item may be tracked by a product identifier, such as a stock keeping unit (“SKU”) identifier. A SKU identifier typically identifies an item/product (i.e. a plurality of items/products having the same property or properties), but does not uniquely identify each item/product. Accordingly, as used herein the term “identifier” encompasses unique identifiers, as well as more generic item/product specific identifiers, and identifiers that include both unique identifiers and item/product specific identifiers.

An identifier may take any of a wide variety of forms, but in one embodiment is a numerical or alpha-numerical serial number. Alternately, the identifier may take the form of, or be embedded in, a printed bar code that is scannable/readable/recognizable by a standard bar code reader, and which, when scanned, conveys the item number/serial number/unique identification information, or other information. The identifier may also, or instead, take the form of or be embedded in a RFID tag, readable chip or memory which, when read (i.e. by a RFID reader, computer or the like), conveys the relevant information. The identifier can also take any of a variety of other forms, utilizing coded and/or uncoded formats.

b. Item Identification

When an item is received by a user 14 from a supplier 20, the user 14 may input the identifier for that item into its personal electronic device 18/computer 16 for uploading to the host 12/host computer 10. In one case the identifier/identification information can be obtained by scanning the associated bar code, reading the RFID tag, etc. For example, in the embodiment shown in FIG. 1, the identifier/tag 22, embodied in, or in the form of, a bar code, is carried on packaging 24 and scanned by the personal electronic device 18. Various other manner of inputting the identifier, such as the use of optical character recognition, may be utilized. The information encoded in the identifier 22 is then uploaded to the user computer 16 and/or host computer 10.

In another embodiment, rather than being scanned individually, each identifier 22 may take the form of a removable label, tag or the like. In this case, when the user 14 receives the item, the identifier 22 is removed and transferred to a separate sheet or repository for collecting identifiers. The user 14 can then later enter the received item information in a batch form via the personal electronic device 18/computer 16 for uploading to the central computer 10.

Various other information relating to the item, besides its identifier, may also be tracked. For example, information relating to its physical attributes (i.e., in the case of an IOL, the diopter of a lens), an item description, date of creation, expiration date, supplier, materials, quantity, cost, size, compatible equipment (i.e. an IOL inserter that is compatible with an IOL), ancillary or related items typically used with the item, or other data or information relating to a property or characteristic of the item may be tracked. In one case, this information may be embedded in, or combined with (i.e. in the same bar code or tag) the identifier 22. Alternately, this information may be presented entirely separate from the identifier 22, such as on the packaging 24 of the item, in the form of human readable or machine readable data. In this case, the user may scan another bar code, or read the packaging, etc. to extract the desired additional information and enter it into the system.

Each supplier 20 may have its own data format in which the identifier 22 and/or other information is presented. In this case, when the user scans the identifier 22 or other information, the format of the scanned information can be compared to known formats to find a match, after which the relevant information can be mapped and extracted. In some cases, an unrecognized format for the identifier, or other information, may be encountered. In this case, the user may be notified so that corrective action can be taken. Alternately, or in addition, the system and method may be configured such that if an unrecognized format is encountered, the system and method attempts to recognize and/or accommodate the unrecognized format by comparing the unrecognized format to known existing formats, selecting the closest logical match, and mapping attributes of the known format to the new/unrecognized format. In this manner, once the unrecognized format is mapped, relevant information can be extracted and processed. When an unknown, but mappable, data format is encountered the user may be notified so that the user can review and verify the appropriate extracted information.

The system and method includes an inventory function/module and/or an inventory database in which information relating to all of the inventoried items are stored and tracked, for example, at the host computer 10. The inventory module may track uniquely identified and/or non-uniquely identified items. The various properties characteristics of the items outlined above may be stored and tracked in the inventory module, although the user may determine the extent to which various information is tracked.

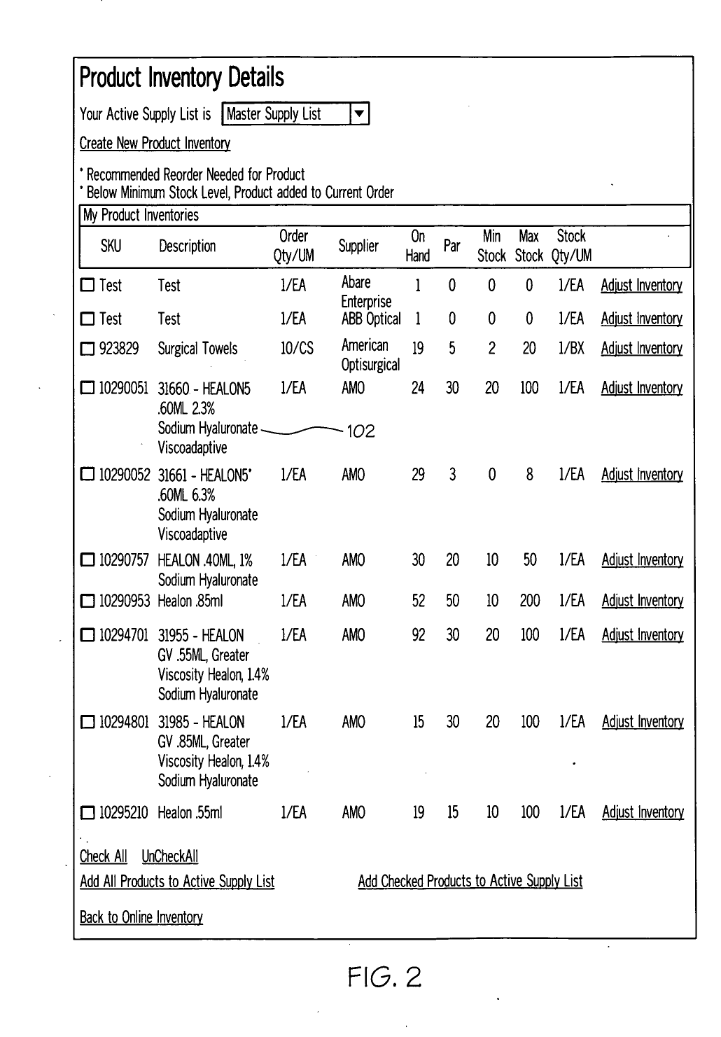

The inventory module and database (as well as the other features and modules described herein) may be easily navigable using standard windows/browser features, such as clickable links/items, scroll bars, hyperlinks, navigation buttons, etc. For example, the user may navigate to the inventory module such that a list of all inventoried items is presented. FIG. 2 is a screen shot showing inventory details for certain medical supplies for a particular user. As can be seen, the system provides information relating to the various items, such as SKU, description of the item, the order quantity/um (i.e. order quantity per unit measure), identity of the supplier, on hand values, par, minimum, maximum, and stock quantity/um (i.e. stock quantity per unit measure).

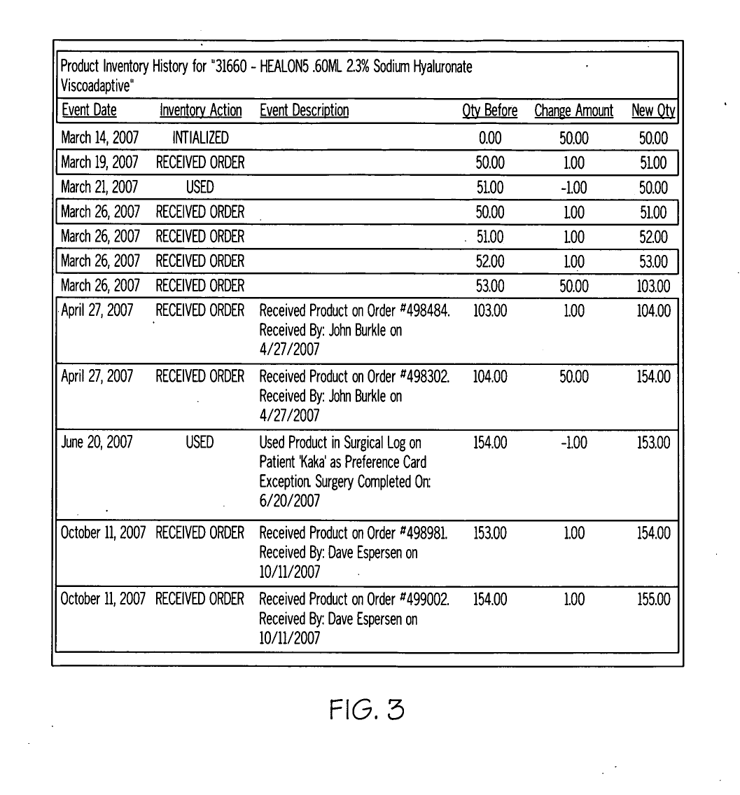

If more information about a particular item is desired, a link associated with that item can be clicked or otherwise activated to provide additional details. For example, if more detail about the item “31660-HEALON5 0.60 ML 2.3% Sodium Hyaluronate Vicscoadaptive” (the fourth item listed on FIG. 2) is desired, the user can simply click upon that text 102. A resultant screen shot is shown in FIG. 3. In this case, more detailed information relating to the item of interest is provided, including its inventory history, such as details regarding when and in what quantities the item was ordered, received, used, or reconciled (i.e. during inventory correction). Particular descriptions relating to each event in the inventory history may also be provided.

c. Inventory Levels

As noted above and shown in FIG. 2, the system and method may track the amount of items in inventory, including minimum, maximum, and par values for each item. Par value of a particular inventoried item represents the value at which it is recommended inventory for that item be maintained. More particularly, par value may be the value that maintains the best balance between having sufficient inventory on hand to accommodate known or predicted usage, against the downside of carrying too much inventory (i.e. reduced storage space, costs of shelf spaced, increased risk of loss and potential expiration of items, etc.). The minimum value for an inventoried item is the lowest level at which inventory is desired to be maintained, and represents the value at which immediate replenishment may be needed. Conversely, the maximum value for an inventoried item designates the highest levels of a particular item that is desired to be stored due to limited storage space, increased shelf costs, increased risk of loss, potential expiration of items, costs of unused inventory, etc.

When inventory levels for a particular item drops below par level, the system may send a notification to the user. The notification can take any variety of forms, such as a notification window when the user next logs on to the system, or an email, facsimile, text message or the like. The notification may include a link which, when clicked, directs the user to an order form that can be utilized to order additional supplies to replenish inventory, as will be described in greater detail below.

When inventory levels for a particular item drops below minimum levels, the system may consider that the inventory for that item is at an emergency level (i.e., the ability to complete scheduled surgeries or procedures may be in jeopardy). In this case the system may send a notification in the same manner described above with respect to par levels. However, in this case, the message may be sent with an indication of greater urgency, and/or other steps may be taken (i.e., more urgent terminology or formats are utilized, followed by a phone call or fax to the user, etc.). When inventory levels for an item exceed the maximum level, the user may be similarly notified, and, the system may suggest that excess items be returned to the supplier, as will be described in greater detail below. In all three cases, a link or form, optionally with suggested ordering/return levels, may be automatically provided to the user.

The minimum, par and maximum levels may be set by the user based upon its experience, analysis or the like. In addition, the system and method may continuously monitor inventory levels over time and conduct its own analysis to suggest starting levels and/or modifications to the minimum, par and maximum levels. For example, if the system notes that a user consistently falls below par and/or minimum levels for a particular item, the system may suggest raising the par and/or minimum level and provide a new suggested value. The user may then be cued to accept, reject or modify the proposed new minimum inventory level. For example, when suggesting a new par level, the system may examine the user's usage pattern and average delivery time for a particular item, and recommend that par level be set such that the amount of items on hand is always just sufficient to support all scheduled surgeries.

In addition, the system and method may be configured to track trends in the user's inventory usage, and accommodate such trends into its predictive analysis. For example, the system may note an increase in implant surgical procedures at the beginning of a calendar year (for example, when medical reimbursement accounts are replenished). In this case, the minimum, par and maximum levels may be correspondingly adjusted, or suggested to be adjusted, in the months preceding the beginning of a given calendar year to build up inventory to accommodate a historical or anticipated increase in implant procedures. The system can also track and analyze trends for individual doctors (i.e. based upon vacation patterns), seasonal variations, fluctuations in economic conditions, cost of materials, promotional campaigns, etc. In this manner, the system and method provides a suggestive tool that can be utilized by users to predict demand and maintain inventory at its most efficient levels.

In yet another embodiment, the predictive elements of the system and method also track expiration dates of inventoried items against projected use. In this manner it is possible to predict when inventory levels for a particular item will fall too low due to projected use while considering the additional factor of upcoming expirations. The system and method can also provide an alert to the user if later expiring items in inventory are being used before earlier expiring items. This can allow the user to correct its inventory usage to increase efficiency.

As noted above, and described in greater detail below, when inventory drops sufficiently low or high, the user may be directed to an ordering form/return form. Alternately, the user may grant the system and method authority to automatically order items and/or institute returns based upon user-defined criteria, and, if desired, user approval.

4. Supply Ordering

a. New Product Inventory

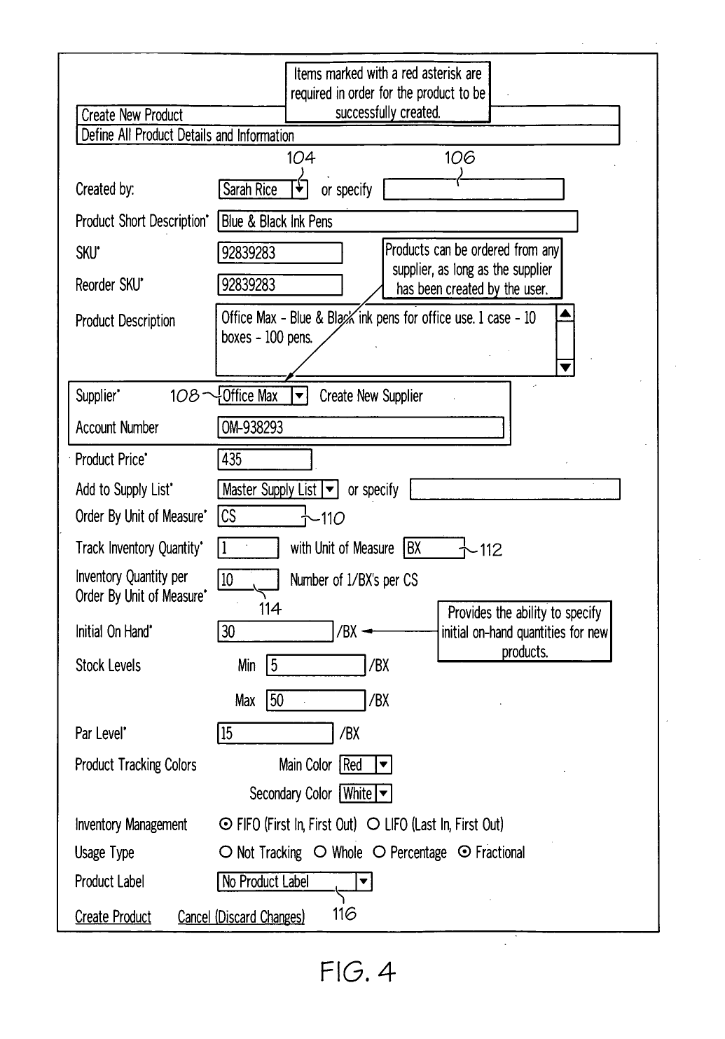

In order to set up a new product in its inventory, the user navigates to, or is directed to, a “Create New Item Inventory” form, as shown in FIG. 4. As can be seen, a wide variety of information relating to the item to be created in the system and method is presented and/or can be entered. For example, the identity of the person creating the data entry can be selected via the drop down selector 104 that allows the selection of pre-defined individuals. Alternately the identity of the person can be manually entered via a user free form text input box 106.

The new item form may also include fields for a user to enter an item description, SKU/model information, stock/quantity information, order/quantity information, item price, the identify of the supplier(s), the account number for the user with respect to each supplier, units of measure, units of measure for inventory tracking purposes, the ratio between the units of measure for the order and units of measure for inventory tracking purposes (as will be described in greater detail below), on-hand levels, par levels, inventory tracking parameters, usage tracking levels, etc.

The form of FIG. 4 also allows information about the inventory status of the item of interest to be entered. For example, the initial on-hand level (i.e., units in stock) may be provided. Minimum, maximum, and par stock levels are entered, as set by the user, possibly with suggestions from the system and method.

The system and method may provide functionality allowing the user to define which suppliers the product can be ordered from. In the example depicted in FIG. 4, a supplier drop down menu 108 is used to define suppliers from which the particular item may be ordered. The account number utilized by the selected supplier for the user may be automatically provided or manually entered. Various other information related to the various suppliers, such as their address, items offered, requirements for orders and returns, billing procedures, contact information, etc. may be automatically provided or manually entered.

The form of FIG. 4 may also be used to define the unit of measure for orders for that particular item. In the example shown in FIG. 4, the units 110 in which the item (in this case—pens) is ordered is by the “case.” The form may also have a location 112 in which the user can specify the unit of measure by which inventory quantities will be tracked after receipt. For example, in this case, the pens are tracked in inventory by the box. The user may be able to specify the relationship between the various units. For example, in this case, at the field 114 labeled “Inventory Quantity per Order By Unit of Measure” it can be seen that there are ten boxes per case.

In this case the system and method allows the user to track inventory in terms of the units actually stored/used (boxes in this case), while tracking purchases in terms of the units actually shipped. The system and method may thus be able to directly convert the number of received units into the number of inventoried units based upon the specified ratio. Information relating to the ratio of received units to the number of inventoried units may be stored in the host computer or extracted from information associated with the item or obtained from the supplier/manufacturer. In this manner, the system and method utilizes what may be termed “reorder unit measure/tracking unit measure” logic (also termed “RUM/TUM” logic herein) to ensure accurate tracking of items as ordered, stocked, and actually used.

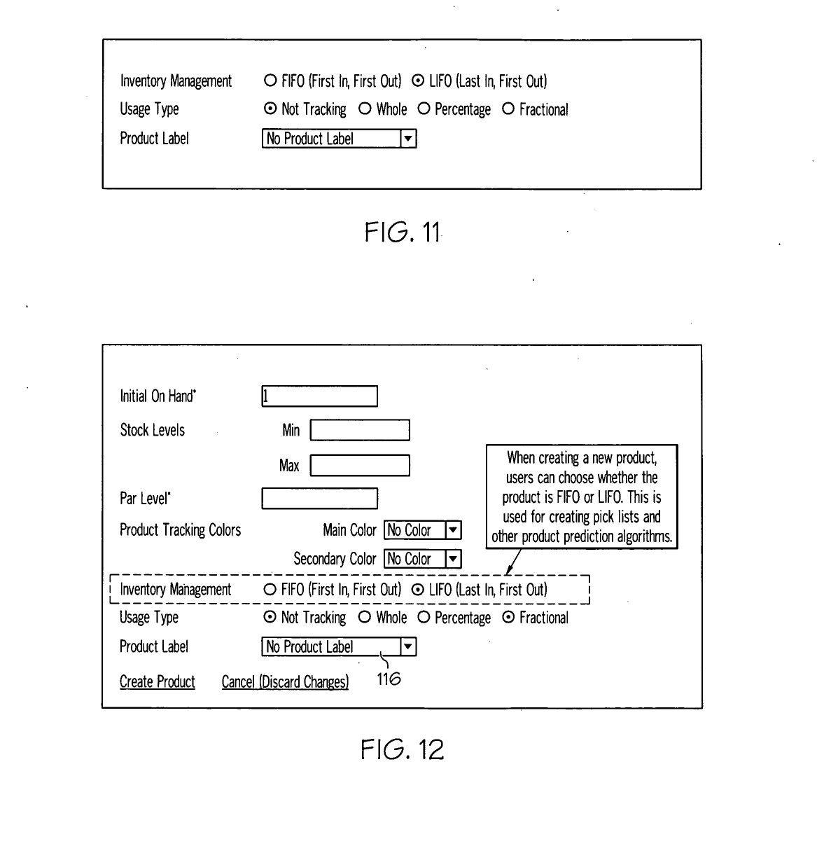

When a new inventory record is created, the user may be cued to enter a usage parameter or usage type for that item (see FIGS. 4 and 11). The usage parameter relates to the smallest unit of measure by which an item is tracked. For example, in the illustrated embodiment the specified usage parameters are “not tracking,” “whole,” “percentage,” or “fractional.”

A “whole use” item is one that is wholly used or consumed in a single usage. For example, surgical drape, or an implant, may be appropriately designated whole use items. A percentage or fractional usage item may have multiple uses before the item is used up, or before the end of its useful life is reached. For example, a bottle of saline may be projected to have about twenty “uses,” a scalpel blade may be determined to have six “uses” (i.e., the blade is deemed to have lost sufficient sharpness after six cuts), etc. An item can be identified as either a percentage or a fractional usage item depending upon the preferences of the user, industry custom, manufacturer specifications, etc. Finally, a non-tracking item is an item which is not used or consumed, or may not have any particular or predefined usage or life (i.e. a surgical gurney, a pump, etc.)

The usage parameter for an item, along with the number of uses (if applicable) may be automatically extracted from the information associated with that item (i.e., from a scanned bar code), or from a database that is maintained by the host, or a database maintained by the associated manufacturer or supplier, etc.

The user also has the option of assigning an accounting tracking designation for its items. The accounting tracking designation determines whether the inventory for that item will be tracked using FIFO (first in, first out), LIFO (last in, first out), or some other accounting method (see FIGS. 4 and 12). The FIFO/LIFO flag is used by the system and method to determine which particular item (with its own associated data, such as pricing, date of acquisition, expiration date, etc.) is to be subsequently utilized in procedures and for accounting and inventory purposes.

The user may also have the ability to control the manner in which inventory is tracked in its system. For example, an item that has been ordered, but not shipped, may or may not be counted in the user's inventory, depending upon the user's preferences. The same may apply to items that are shipped but not received (i.e. in-transit items), items that are received but not yet stored or entered into the system, etc. Conversely, inventory can be reduced at the occurrence of various events as determined by the user. For example, inventory may be reduced when items are marked for use in a procedure, or marked for return or upon verification that the item has been actually used or returned, etc. Thus the system and method has the flexibility to track inventory in a manner that matches the user's preferences. The manner in which inventory is tracked will effect the user's accounting, case costing, item reordering, and the like.

After the desired information about the item, including an item/product description, usage type, FIFO/LIFO flags, manner of tracking inventory, etc. are entered into the user's computer, the information relating to the received items is uploaded from the user's computer to the host computer by computer communications or the like. The received information is stored by the host computer for that particular user in one or more databases. The data may be uploaded to the host computer as soon as the data is received in the user's computer (i.e. if there is a connection between the user's computer and the host computer). Alternately, the data may be stored or cached in the user's computer until it is uploaded to the host computer (i.e., when an internet connection is established, or at periodic intervals, etc.), or simply stored and utilized on the user's computer without uploading.

b. Ordering Options

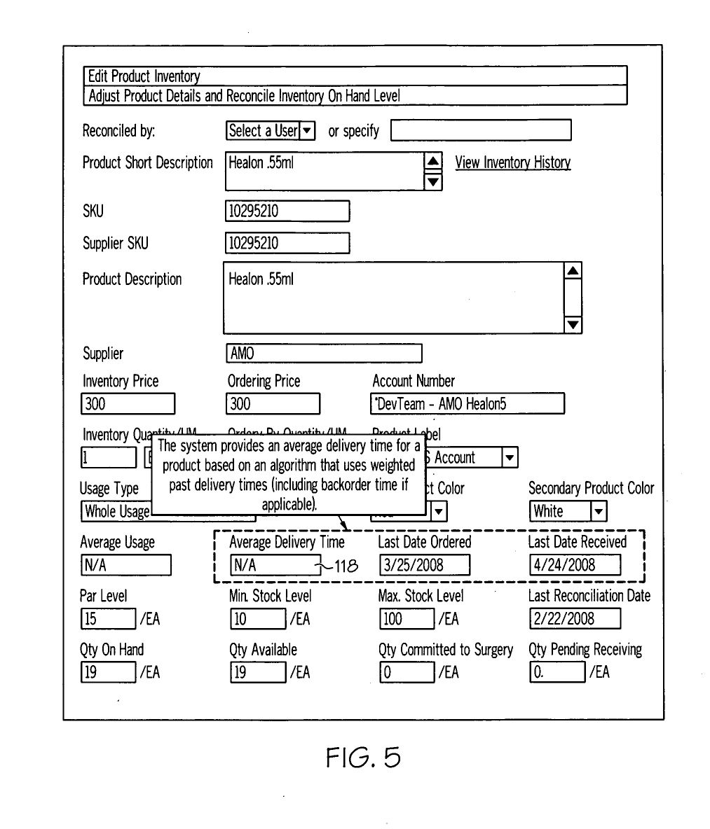

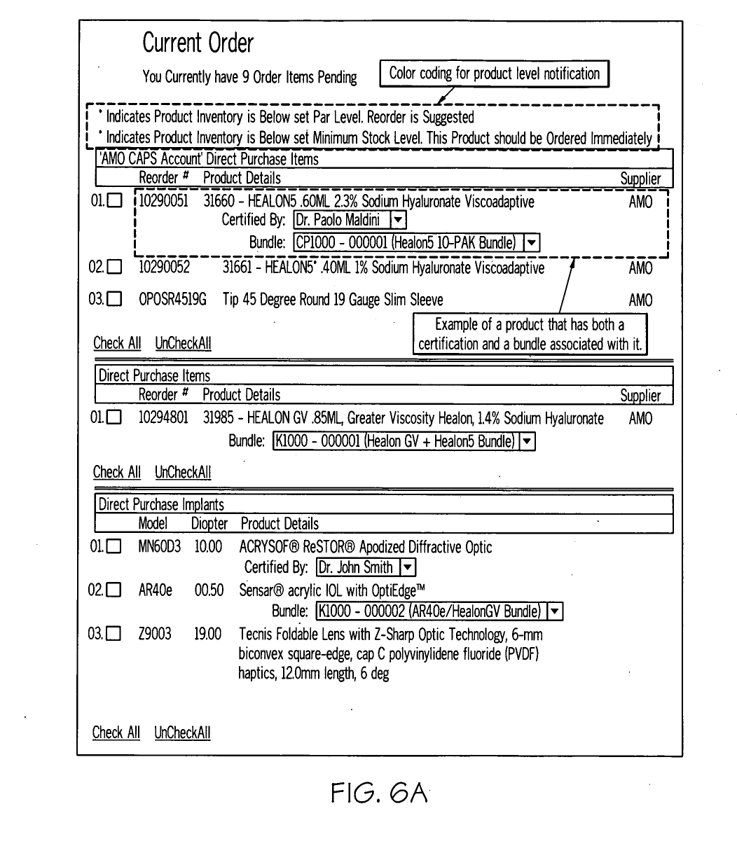

In order to request new items to replenish its inventory, the user navigates to, or is directed to, a form for ordering new products (an order or reordering form), which may look similar to, or require similar information to, the form shown in FIG. 5. As noted above, when inventory levels drop below minimum (and/or par levels) the system may send a notification to the user and provide a link which, when clicked upon, directs the user to an ordering screen, such as the one shown in FIG. 5 or similar thereto. Alternately, the user may navigate to the ordering screen on its own.

The item to be ordered can be identified in any of a variety of manners, such as by searching for a particular item, selecting from recently or commonly ordered items, navigating through a hierarchy-based system relating to item features or functionality, scanning the identifier 22 on the item to be created/ordered, or in various other manners. Once the desired item is displayed, the user can simply check or click upon the item that is desired to be created/reordered, thereby resulting in the on-line fillable reorder form, as shown in FIG. 5.

The reorder form allows the user to select the supplier, the number of products to be ordered, the manner of delivery, etc. Minimum, maximum, and par levels of inventory as well as on-hand numbers may be displayed in the reorder form to aid the user in determining how many items to order. Moreover, as noted above, the number of items to be ordered may be suggested by the system/method, which can take into consideration scheduled procedures and various other factors. For example, the system and method may suggest ordering related items based upon upcoming scheduled procedures, suggest ordering items based upon the levels (or projected levels) of items compared to par or minimum values, or based upon a desire to maintain a particular ratio of items relative to other (related) items.

The system may track various information about the delivery time for a particular item to aid the user in determining how much of the item should be ordered, and what sort of delivery method should be required. For example, when an item is selected for ordering, the average or median delivery time for that item may be displayed in field 118, as shown in FIG. 5. The average shipping time can be broken down by item and/or supplier to provide more detailed information to aid the user. In addition, various methods of calculating the “average” delivery time may be utilized. For example, more recent delivery times can be weighed more heavily in the calculating the “average” delivery time to increase predictive accuracy. The average delivery time can also take into account delays to due backordering of items. In addition to displaying the average delivery time, the most recent delivery time can be displayed.

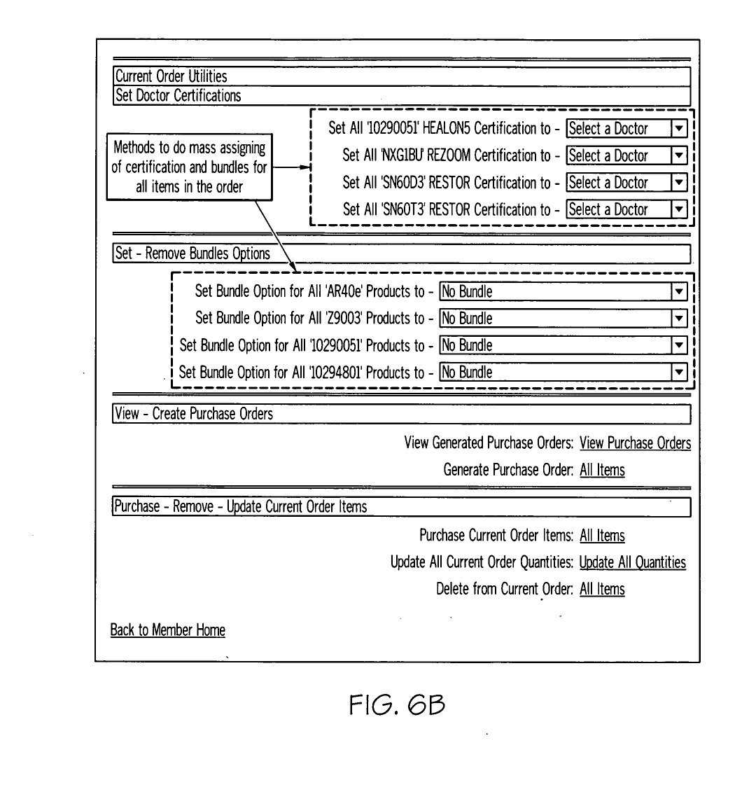

When ordering an item, a “Current Order” form such as that shown in FIG. 6, may be displayed concurrently with, or in sequence with the form shown in FIG. 5. This form displays pertinent information relating to the item to be ordered, and allows the user to manage and edit the order and select particular options. All of the ordering forms and sub-forms may utilize color coding to indicate when an item is below par level or minimum level, or above maximum level. In one particular embodiment, the details related to certain items are provided in green to illustrate that inventory levels are below the minimum for that item and that the item should be ordered immediately. In addition, other colors may be used to convey information that inventory levels of a particular item have dropped below par levels, or exceeded maximum levels. In addition, it should be understood that this coloring scheme can be associated with the various item throughout the system and method and in the various screens utilized therein.

As shown in FIG. 6, the Current Order form may segregate various discreet portions of the order as desired to aid the user. For example, the first grouping of items of the order shown in FIG. 6 is identified as a “CAPS Account Direct Purchase Items.” In this case that particular part of the order is identified as being made under the terms of a capitated agreement (“CAPS”), which is a pre-defined ordering or contractual arrangement between the user and a particular supplier specifying that the user will buy or otherwise acquire a particular volume of items under an agreed-upon pricing model. In this manner, the segregation of CAPS purchases from non-CAPS purchases allows the user to ensure that orders are made in the desired manner with respect to its various CAPS agreements. Besides, or in addition to, arranging orders based upon their status as a CAPS or a non-CAPS order, the pending order can be broken out, or arranged, by a variety of classification criteria, such as shipping method, shipping location, purchase order number, etc. In addition, the system and method may be used to place orders with nearly any supplier which provides goods or services, regardless of whether the user has a predefined contractual relationship with the user, so long as the user can establish a communication link with the supplier and provide the necessary information for an order.

Items may be ordered on a consignment basis, depending upon the agreement between the supplier and user/parent facility. Alternately, if desired, items may be directly purchased/acquired by the user from the supplier. These parameters can be specified on the order form filled out by the user, and/or may be noted by the user at the time of receipt of the ordered items. It can be important for the system and method to track the status of particular items as being either purchased or on consignment. For example, if an item is on consignment, the system and method need to know this to notify the supplier when a consigned item is utilized.

The user can specify various differing shipping methods within an order (i.e. request expedited shipping for particularly high priority items). The user can also specify differing ship-to addresses for various items within a single order so that various items are sent to their desired destination. The user can also specify particular accounting tracking designations, usage tracking designations, labels, etc. for particular items at the time of ordering.

When ordering items from a particular supplier, the user may wish to bundle items, or may have an understanding or agreement with the supplier that certain items can, should or must be ordered on a bundled basis. Bundled items may be items that have complementary functions, uses, billing relationship, or the like. For example, if an IOL is ordered by the user, then an implant inserter, which is used to aid the physician in implanting the IOL, may be bundled or suggested to be bundled. Suppliers may provide a price break when items are ordered on a bundled basis.

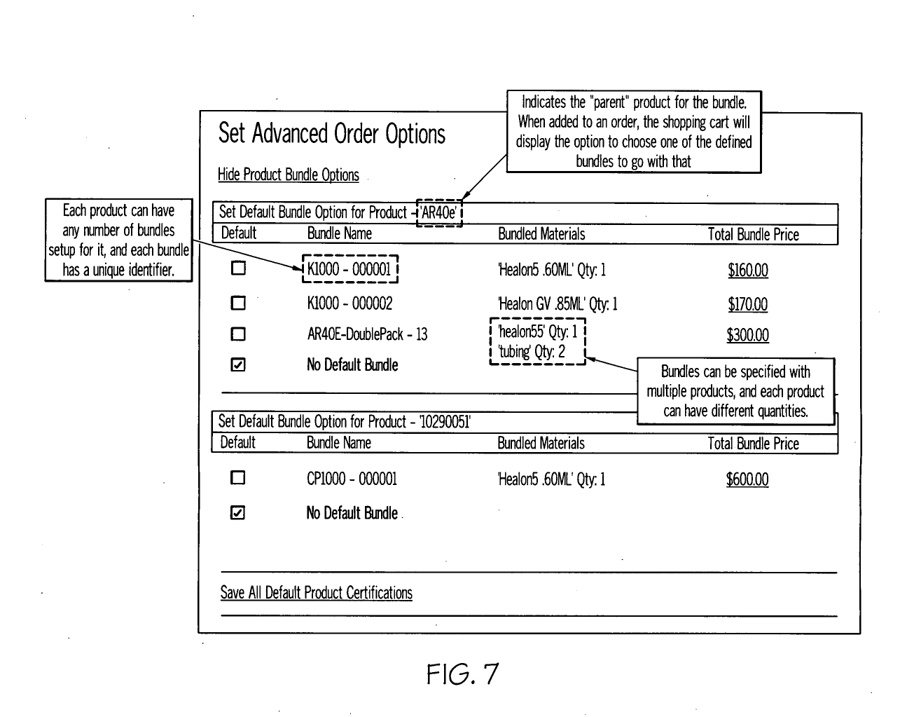

In this case, one of the items (i.e. the implant, in this case) is designated as the “parent” item, and one or more “complementary” items that can be bundled with the parent (i.e. the implant inserter, in this case) are automatically populated with the parent item or suggested to the user (see FIG. 6; see also FIG. 7), possibly in a new screen for the user to view, modify and/or select. Alternately, a clickable button, drop-down or link may be provided so that the user can see what bundling options are available for a given item. The quantity of complementary items to be ordered may be able to be automatically adjusted to maintain a desired ratio with respect to the quantity of the parent item. In some cases, the ratio of items in a bundle may be set by the supplier and not modifiable by the user. In other cases, the number of complementary items can then be adjusted by the user to the desired level before proceeding to finalize the order.

The existence of bundles, the type of items to be bundled, and the ratio of items in a bundle may be initially set or suggested by the system and not adjustable by the user. Alternately, or in addition, in some cases the user may be able to adjust the properties of the bundles, create new bundling arrangements, de-activate bundling arrangements, etc., to reflect the user's desires, the supplier's desires, and/or a change in agreement between the supplier and user. In some cases, a particular item may only be able to be ordered from a particular supplier on a bundled basis (such as when the item is physically bundled with other items, or when the customer has entered into an agreement with a supplier, etc.) in which case the ability of the user to de-bundle the items may be disabled. The system may also allow the user to view the history of past bundled orders to aid in the ordering process.

Bundles may also be defined for the purposes of adjusting inventory. Thus if, for example, the user indicates that a certain number of a particular item is going to be used, the system may assume, or suggest, that a certain number of related items are also going to be used (i.e. an indication of use of an IOL in a procedure may cause the system to assume or inquire whether an IOL inserter will also be used). In this manner, defined product relationships can aid in tracking inventory, automatic or suggested reordering levels, etc.

The system and method may also have the ability to track what sort of products are typically ordered together and/or utilized together (or in close proximity). In this case the system and method may suggest that bundling relationships be created for ordering purposes, purchasing contracts, inventory tracking etc. In this manner the system and method may be able to recognize and suggest relationships between items that are not necessarily apparent to the user.

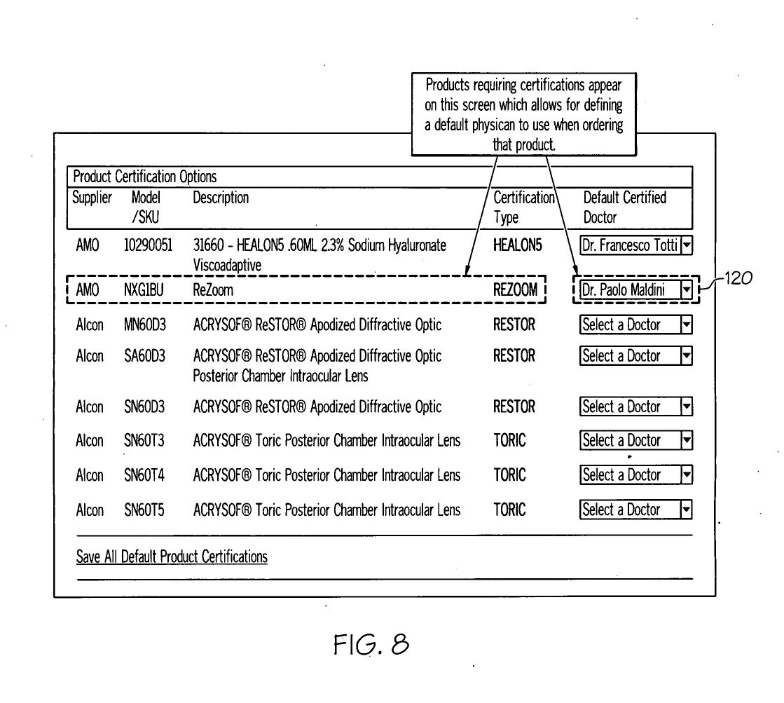

The system and method may also be set up to provide additional restriction upon the ordering of items, as set up by the user, host, or based upon supplier requirements. For example, certain suppliers may dictate that certain items (such as pharmaceuticals, expensive items, items prone to misuse, items which require training, items which require certification under a CAPS agreement, etc.) can only be ordered by certain individuals, such as certified physicians. Certified physicians may, for example, be certified by the manufacturer or distributor, by a government or administrative agency, or another institution. As shown in FIG. 8, when such a restricted items is indicated to be desired to be ordered, a drop down menu 120 may be provided which lists physicians that are certified by the supplier to order that item. The drop down menu may be automatically provided when an item requiring physician certification is indicated to be desired to be ordered (see also FIG. 6). In this manner, user certifications are transferred to suppliers as part of the ordering process.

In certain cases, once the certified physician is identified, the user may be required to authenticate the transaction via security measures (such as entering a proper password, personal identification, physical token, biometric id, or the like) in order to proceed with the order. Once a user is authenticated, if desired, the identity of the individual may then be cross-referenced across a list of authorized individuals maintained by the operator. In this manner the system and method may be operated to ensure that access to restricted items is controlled as required by the supplier, and the ordering process is streamlined by obtaining the required certification at the time of ordering. In one embodiment the authenticated physician's electronic signature is utilized to authorize orders. Certified user management can be implemented by setting up a profile for each certified individual for that user and specifying which restricted-use items that certified user is able to order.

As noted in the exemplary screenshot in both FIG. 4 (and many others), the system and method provides the ability to track and attribute changes in information to particular individuals. The ability to track the identity of the person who changes a given record is useful in auditing and capturing errors. In one embodiment the system and method utilizes an identification technique, such requiring a user ID and password, a physical token, a combination of physical token and password, or other means to authenticate and track individual users. In such cases, the identity of the individual changing a given records are automatically captured.

The user's administrator may also be able to assign specific rights to certain individuals, thereby restricting the ability to change or view certain information based on training, security protocols, or other criteria selected by the user. The system and method may store a history of the original value and modified value for all information changed by a given individual for specified time window or quantity of changes. This allows the user's administrator to “roll-back” or reverse changes made by a specific individual if it is later discovered that spurious or incorrect entries were made by a given individual. Details of changes made by individuals can be used by the administrator to document a history of changes that could be indicative of an attempt to cover up malfeasance or other systematic errors. Administrators may also be able to maintain Health Insurance Portability and Accountability Act (“HIPAA”) compliance by tracking which individuals have accessed or received various medical information.

The user's administrator, or individual users themselves, may be able to set up their own preferences in the system. For example a user may be able to specify preferred providers for that user, store a list of products typically ordered, set up particular utilities, etc. Furthermore, the system and method may track certain user tendencies and suggest that certain preferences be created to match the tracked tendencies.

The system and method may be designed to allow the user to assign item tracking colors. For example, certain items, such as sutures, may utilize a color or colors to identify features about the sutures, such as size, manufacturer, etc. The system and method may be configured to similarly assign, or allow the user to assign, a color or colors to a particular item to aid in tracking and identification.

For example, a particular size of sutures may be shipped and/or stored in a box with a purple and green label. The purple and green label may be known in the industry, and by the user, to be associated with a particular size of suture. In this case, the purple and green color scheme may be used throughout the system and method, or otherwise associated with the particular suture and its storage/packaging. For example, any time that particular item is shown in the system and method, that purple and green color scheme may be utilized on the associated text, surrounding boundaries, on a color patch adjacent to the item, etc. The color scheme may also be used in any print outs (i.e. in an inventory reconciliation sheet).

The item tracking color feature allows each item to be easily visually identified and cues the user as to the nature of the item to help to minimize errors. Further alternately, an image of the item's packaging may be included among the information stored within the database and provided to the individual who is taking stock, re-stocking, selecting stock or otherwise using the system and method to assist that individual in properly identifying the appropriate items. These colors and images can also be assigned when the item is initially set up in inventory, as noted above in FIG. 4.

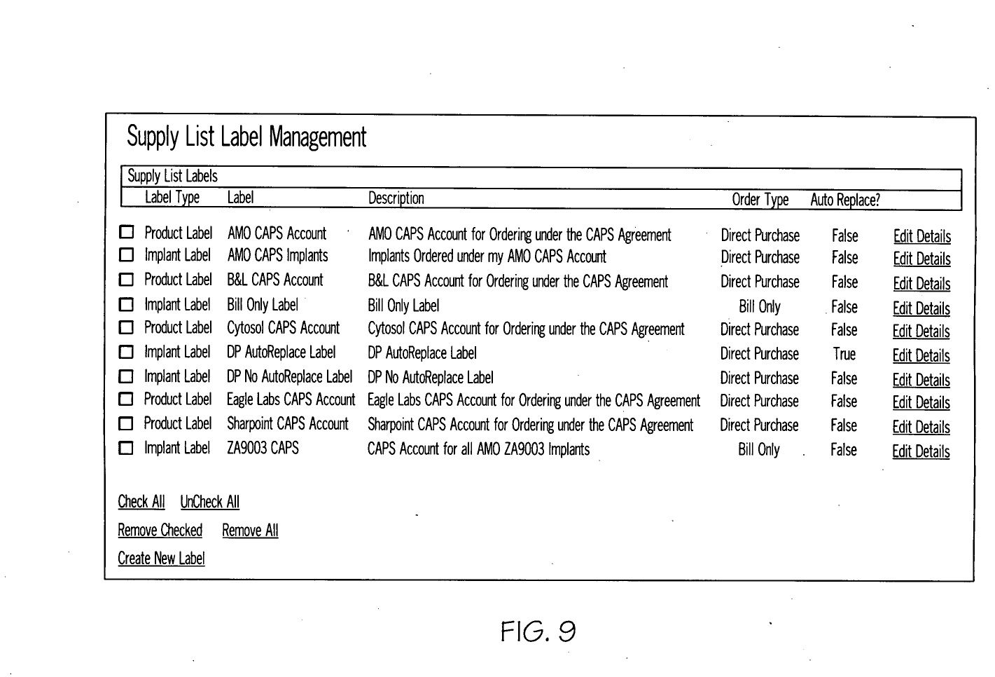

An item label field is provided in the item ordering form, or set up in the item identification form (FIG. 4). Data from this field can be printed out and adhered to the associated inventory, or shelf, or in a book of item barcodes, etc. The label may include a bar code or the like which can be scanned to automatically identify the item associated with the label and/or provide other information, as desired by the user. The labels may aid in inventory reconciliation, pulling inventory items for use in a procedure, or to identify an item for reordering. The labels may also bear the item tracking colors identified above. The labels can also provide additional information about the item, as desired, such as its supplier, whether the item is ordered pursuant to a CAPS agreement, whether the item is ordered as a direct purchase or as a consignment item, etc. As shown in FIG. 9, a list of labels can be generated to aid in supply list label management.

The label feature may also provide the user the ability to generate a user-created identifier that is separate and distinct from the identifier 22 provided by the suppler. The user-created identifier can be useful in tracking sub-units of the item. For example, in the example shown in FIG. 4 and noted above, the supplier may ship items by the box (shown in field 110), and the user may desire to track the items by the case (field 112), and there may be ten boxes per case (field 114). In this case the system and method may allow the user to create ten labels, one for each box, which tie the boxes to their associated case, but allow each box to be individually handled. Accordingly the system and method allows the user to generate, utilize and track user-generated labels to track sub-units of inventory. These labels can also be assigned when the item is initially set up in inventory, as noted above in FIG. 4.

When the user is ordering items (or during initial item set-up), the user may be able to define customized identifiers for the items. For example, rather than identifying an item by its SKU or other identifier, the user may assign a commonly-used name to the item. These customized identifiers may then be used in the system and method when reordering items, when generating preference data forms (described below), or at other points in the system to further improve the user-friendliness of the system.

Thus, it can be seen that the order form and various ordering options allows the user to define items in the system with various details that are used for tracking the items and managing the full life cycle of the ordered items. The order form and system disclosed herein allows the user to define, edit, customize, and set attributes for items as the inventory records are created or changed. The system may be configured such that not all of the fields on the ordering form are required in order to complete an order, although certain fields may be deemed critical, such that an order cannot be placed unless the critical fields are completed. The optional fields thereby provide flexibility to the user in how much or how little information is specified.

c. Supplier and User Relationships

The system and method discussed herein may be configured to accommodate, and reflect, various relationships between the user and supplier. For example, various entities may have a parent and/or child corporate or affiliate relationship with the end user. More particularly, and by way of example, the user may have a parent entity or corporation which owns or controls the user. The parent may own and/or possess certain supplies that are desired to be utilized by the user. The parent entity may be a separate, controlling entity, or may simply be a warehouse. In this case, the system and method may store or access the inventory information of the parent (or other related entities) when the user desires to place an order.

In this manner, when the user places an order for certain supplies, the system may make a query or a suggestion to the user to order supplies from the parent (or other associated or related entity (if such supplies are available)). A preference may be set up for the user to order supplies from its parent (or other associated or related entities), if possible, to provide a cost savings and/or to relieve the associated entity of its excess inventory. Thus, particular suppliers can be specified to have a particular relationship to the user, such as a parent or child facility. In this manner, the “supplier” field in the order form may be automatically populated in some cases.

Moreover, parent supplier facilities may have the ability to oversee and control the purchases/acquisitions of their “child” facilities. For example, parent facilities may maintain a large base of inventory and transfer (or provide access to) items to their children facilities. In this manner, the parent facility may take over the responsibility of ordering items directly from suppliers to leverage its negotiating power and secure bulk purchase discounts. Sibling (or other related) facilities may be able to transfer items between each other, either with or without parent facility oversight.

Moreover, multiple “tiers” of family relationship can be provided in which, for example, a corporate parent facility shares its inventory with other parent facilities, and/or their children facilities, so that all the entities share a common inventory of products and items. In this manner, if a particular item is backordered and the parent and/or a particular child is lacking sufficient inventory, a user can receive items from another related entity to ensure the efficient use of resources.

In some cases, the parent facility may oversee and control all orders or requests placed by child facilities. For example, the system and method may be set up such that authorization from a parent facility is required before a child facility can order items from a supplier. Parent facilities may also have the ability to view and/or control all inventory levels of their child facilities. On the other hand, parent facilities may allow their children facilities to order directly from suppliers as a matter of course, or, alternately, only in particular circumstances (i.e. when inventory drops below minimum levels, or when the parent facility lacks sufficiently inventory, etc.) Parent facilities may also have the ability to place orders as drop shipments sent directly to their children facilities.

d. Order Processing

Once the order form (i.e. of FIG. 5) is filled out, the order may be sent to the host 12 immediately after completion, or various orders may be aggregated for a particular supplier 20 and sent periodically to the host 12 in a batch manner to secure favorable prices or other conditions. The orders may be sent via secured computer communications, such as by encrypted communications or by email, fax, EDI, and the like. A history of each order is stored automatically so that orders and their details can be tracked.

Once the order form is filled out by the user 14, the relevant information may be sent to the host 12, which then forwards the relevant information to the supplier 20. The host 12 may immediately forward the order to the supplier 20, or may aggregate orders from various users 14 before sending them to the supplier 20. In processing the order information, relevant information may be extracted from the order form and placed in a form or format acceptable for ordering with the supplier 20. In one case, the relevant data may be placed in an EDI format and electronically sent from the host 12 to the particular supplier 20. In this manner, the user 14 needs only to fill out a single on-line fillable form, and the host 12 or host computer 10 automatically identifies and contacts the supplier 20 and forwards information in the appropriate format, and via the appropriate EDI conduits (where necessary). The system and method can thus accommodate disparate requirements by the differing suppliers 20, such as the use of differing purchase orders, forms, and required information by differing suppliers. In this manner, the user need only to become familiar a single interface, and fill out a single form, to order items from multiple suppliers 20, with particular variations within an order.

Moreover, as shown in FIG. 1, rather than sending the order information to the host 12, which then re-transmits the relevant information to the supplier 20, the order may be sent directly from the user 14 to the supplier 20. In this case, the software provided by the host 12 and utilized by the user 14 may automatically carry out the steps specified above (i.e. extracting relevant information from the order form and arranging the information in format acceptable for ordering with the supplier 20, etc.). Rather than electronically forwarding data, in one case the system and method prepares an order form for the desired supplier 20 in the form of a printable document that is suitable for faxing or other transfer by the user 14 to the supplier 20.

Once the order is received by the supplier 20, the supplier 20 pulls the items identified in the order, in the specified quantities, and ships the order to the address and in the manner specified by the user 14. Once the ordered items arrive at the user, the items or their packaging can be scanned and uploaded into the host computer 10 to update the user's records, as noted above and described in greater detail below.

Thus it can be seen that the system and method described herein provide a highly flexible, user-friendly and intuitive system for ordering items. The ordering forms allows the user to enter various information, while simultaneously providing information (such as current inventory levels, suggested levels, average ordering time, etc.) to aid the user in determining parameters of the order (i.e. amounts, delivery method, etc.) Various information may be automatically populated into the fields to aid the user in filling out the form. Certain optional fields are provided so that the user is provided flexibility in how much, or how little, information is utilized and tracked. Once the order is received, the same wealth of information provided in the ordering form is again accessible by the user to aid in receiving and inventorying the received items, as will be described below.

5. Item Intake and Returns

a. Intake and Reconciliation

As items and products are received for the various suppliers, they need to be entered into the inventory system so that they can be tracked. For example, when an item is received, its relevant information may be entered into the system manually (i.e. via a keyboard or the like), or by scanning a bar code or RFID or the like, or by having the system parse order information from the supplier or manufacturer to extract unique and non-unique item identifiers, and various other information relating to the item, as described above. The system and method can then incorporate the inputted information and update its records, including automatically adjusting inventory levels, recalculating average delivery times, and the like as desired.

The present system and method may be configured to enable “one-click” receiving of a plurality of items into inventory. For example, when an order is placed through the system and method, the items requested in the particular purchase order can be stored in the system in a receivable page or data form, as noted above. When the order is received from the supplier, the user can simply recall the previously-placed purchase order, and click upon an icon to indicate that the ordered items have been received. Item inventory and other data fields are then automatically updated. In this manner, when an order (i.e. of multiple items) is received, all of the received items can be entered into the system and method and automatically recognized, for inventory/accounting purposes, by the single click of a mouse.

Alternately, or in addition, the supplier may send information regarding the shipped goods to the user. In this case, once the supplier has received the order information, and before or after the order is compiled and sent, the supplier may send a shipping notice or the like which includes pertinent information about the goods in the order. This information may be sent by computer communications or otherwise, and is typically sent in advance of the receipt of the items by the user.

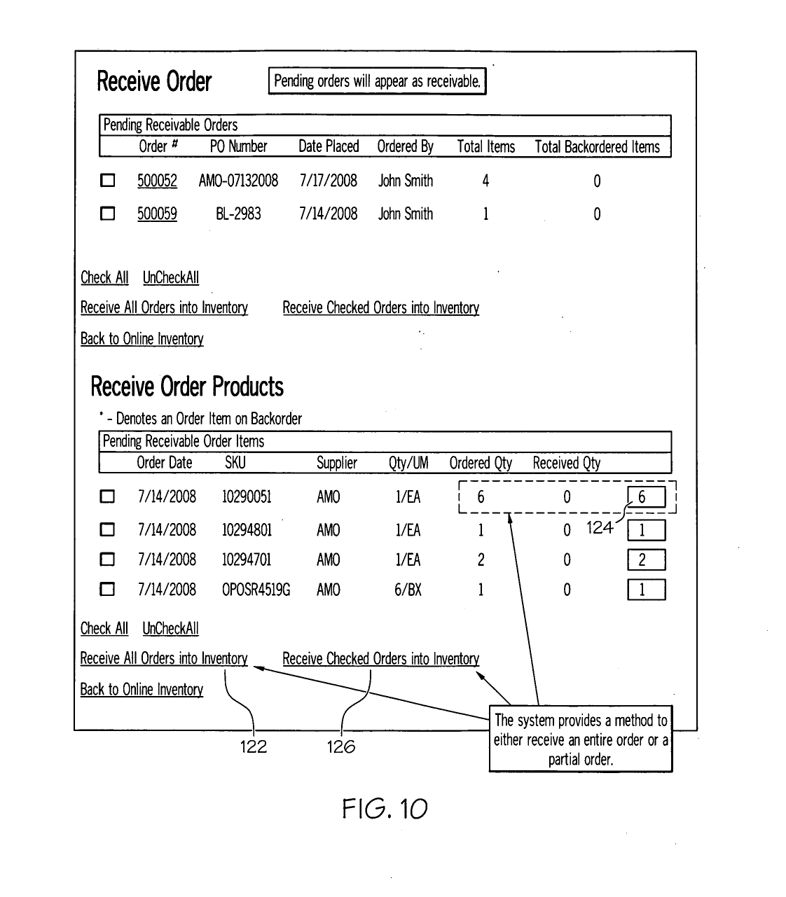

FIG. 10 illustrates a screen shot that may be recalled when an order of items is received by a user from a supplier. The order information may be recalled by the user manually navigating through previous orders or shipping notices; or the system may present a list of orders or shipping notices projected to be received on that particular day. Order information may also be recalled by scanning a bar code on a particular received item, which causes the system and method to recall which outstanding orders or shipping notices are pending for the scanned item, and present the potential orders or shipping notices to the user for selection.

Once the appropriate order is identified, as can be seen in FIG. 10, the system and method recalls all of the items for that particular order, and displays their SKU, supplier, qty/um (quantity per unit of measure) and ordered quantity. If all of the ordered items are actually received, the user can simply click upon the icon 122 labeled “Receive All Order Items Into Inventory,” (or other similarly worded icon) thereby enabling receipt of multiple items with a single click, without having to manually enter or scan each particular item in an order.

The system and method are also configured to accommodate the receipt of partial orders, when less than all of the ordered items are received. For example, as shown in FIG. 10, when an order is received, the user can then click on the “Received Quantity” input field 124 to reflect the fact that less than all of the particular items for that order have been received. For example, in the highlighted field of FIG. 10, six quantities were ordered; if less than six are received, the number can be adjusted as desired. Once the received number of items is set, the icon 126 for “Received Checked Order Items Into Inventory” (or other similarly worded icon) is then clicked or selected. Thus, even partial orders can be quickly and accurately entered into the system with only a few clicks to adjust for the actual, received amounts. After the order is indicated to be received/entered, serialized item inventory on-hand values are automatically updated. If desired, the user can also specify particular accounting tracking designations, usage tracking designations, labels, etc. for the received items at this time. In this manner the system and method disclosed herein ensures that accurate inventory information is maintained, which can be easily and automatically updated upon the receipt of new items.

The user may periodically (i.e. quarterly, yearly, or at other intervals) conduct an inventory reconciliation by manually inventorying its items actually on-hand. The inventory can also be manually taken at any other desired time, such as when an unusual usage/ordering pattern is noticed, when a real-time picture of inventory is desired, or otherwise. The inventory can be taken with a personal electronic device. In the case wherein the personal electronic device includes a bar-code scanner, RFID tag reader or the like, quick and accurate manual inventory results can be achieved by simply scanning each item.

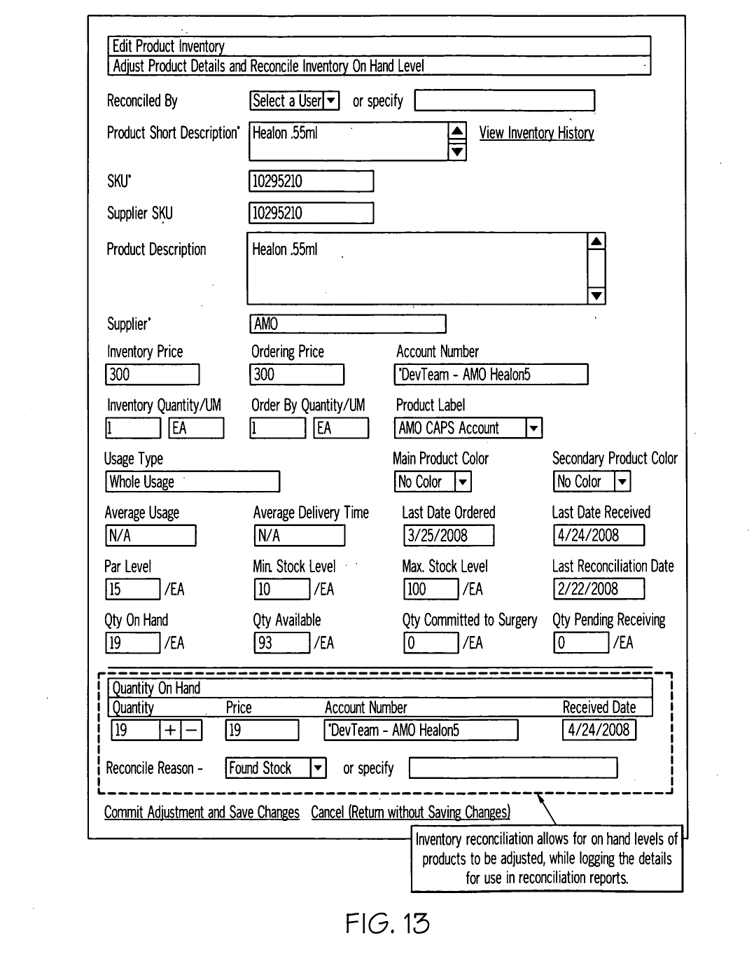

FIG. 13 is a screen shot in which the results of the manual count are entered into the system. Various information, such as the identity of the reconciling user, the item description and identifier, as well as the actual on-hand quantity and the reason for reconciliation may be entered. The results of the manual count are typically set to override the previous values of inventory in the system. Once the results of the reconciliation are input into the system, the system can also quickly and accurately calculate shrinkage, loss of inventory items, etc. Thus, the system and method are set to track authorized and proper usage, but can also accommodate “real-world” situations in which loss, theft, and inaccuracies in data entry can occur, and intelligence regarding such “real-world” events can be collected.

b. Returns

The system and method also aid the user in quickly and easily returning items to the supplier. For example, as noted above, in some cases, exceeding the maximum level of inventory may cause the system to suggest that excess items be returned to the supplier. The user may also desire to return items to the supplier for any of a variety of other reasons, such as defective items, recalls, change in preferences by the user, etc. As noted above, the system already maintains relevant information about the item, such as its SKU, a description of the item, date of order, identity and address of the supplier, etc. Accordingly, when a user desires to return an item to the supplier, the system and method can easily extract the relevant information needed for a return, and generate the necessary paperwork/data such as in a return goods authorization (“RGA”) form or in another particular format required by the particular supplier. The system and method may obtain an RGA authorization code or designation from the supplier, and place the code or designation in the appropriate location of the RGA form to facilitate the processing of the RGA.

In some cases, the supplier may send a plurality of RGA authorizations to the user which are not associated with any particular items, which the user then keeps on hand for future returns. When the user needs to return an item or items, one of the previously-sent RGA authorizations may be accessed and utilized for the return. The system and method may also automatically generate a return merchandise authorization number (“RMA”), or other form, in accordance with the supplier's requirements, if necessary.

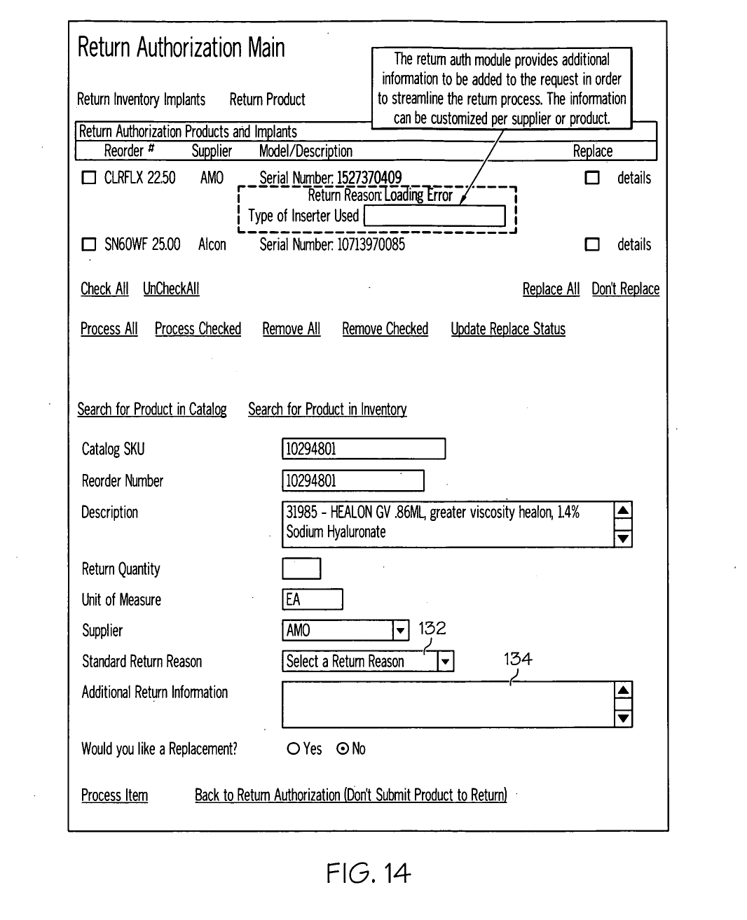

In the case of an item with a unique identifier (or even non-uniquely identified goods), the item identified may be identified or scanned (i.e. with a bar code scanner, RFID tag reader, or other technology described above) to record the unique identifier and extract relevant information about the item. A RGA form or the like may then be automatically populated by the system/method, at least to the extent relevant information is known (as shown in FIG. 14). The user may be required to fill in the reason for the return, indicate whether they would like a replacement, or fill out any other relevant fields that are not automatically populated.

The system/method may then print out an address label, packing list, or other paperwork to aid the user in shipping the items to be returned to the supplier. If desired, the system and method may notify the supplier via computer communications or otherwise that the items are being returned, and provide the supplier with the relevant information. The system may automatically update inventory levels to reflect the reduction in inventory due to the returned items.

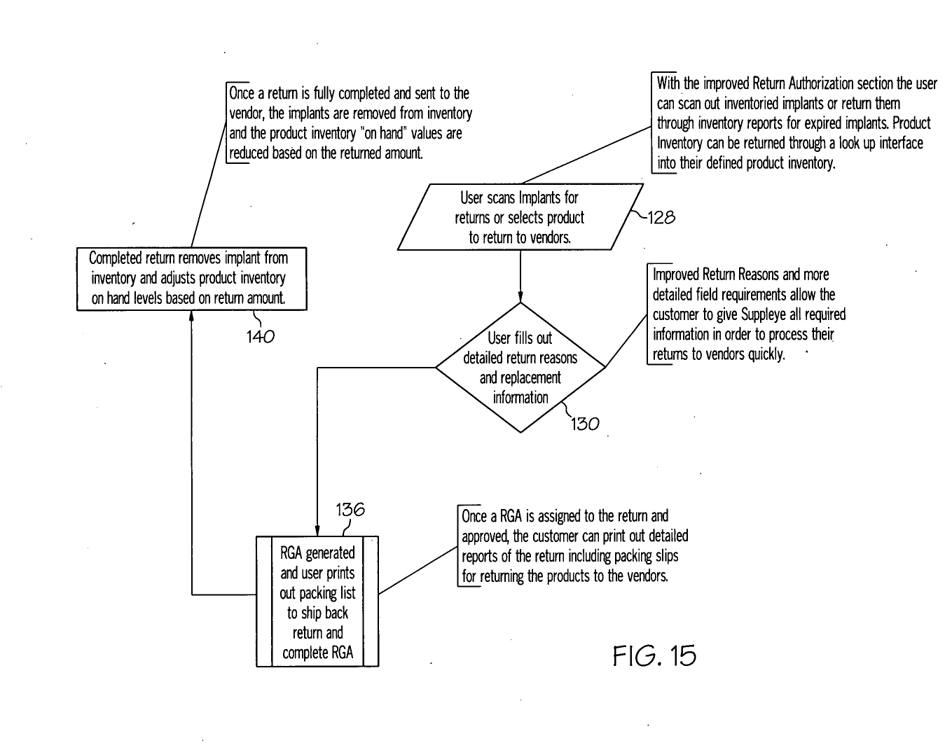

FIG. 15 is a flow chart illustrating the various steps, with annotations, a user may follow to return an item. Thus, it can be seen that at step 128 a user scans the identifier for an item to be returned. At step 130 the user fills out the reasons for return (i.e. fills in fields 132 and/or 134 of FIG. 14). At step 136 the system/method generates an RGA. Once the return is completed, at step 130, the item is removed from inventory and corresponding automatic changes are made.

6. Usage Tracking

a. Preference Data Forms

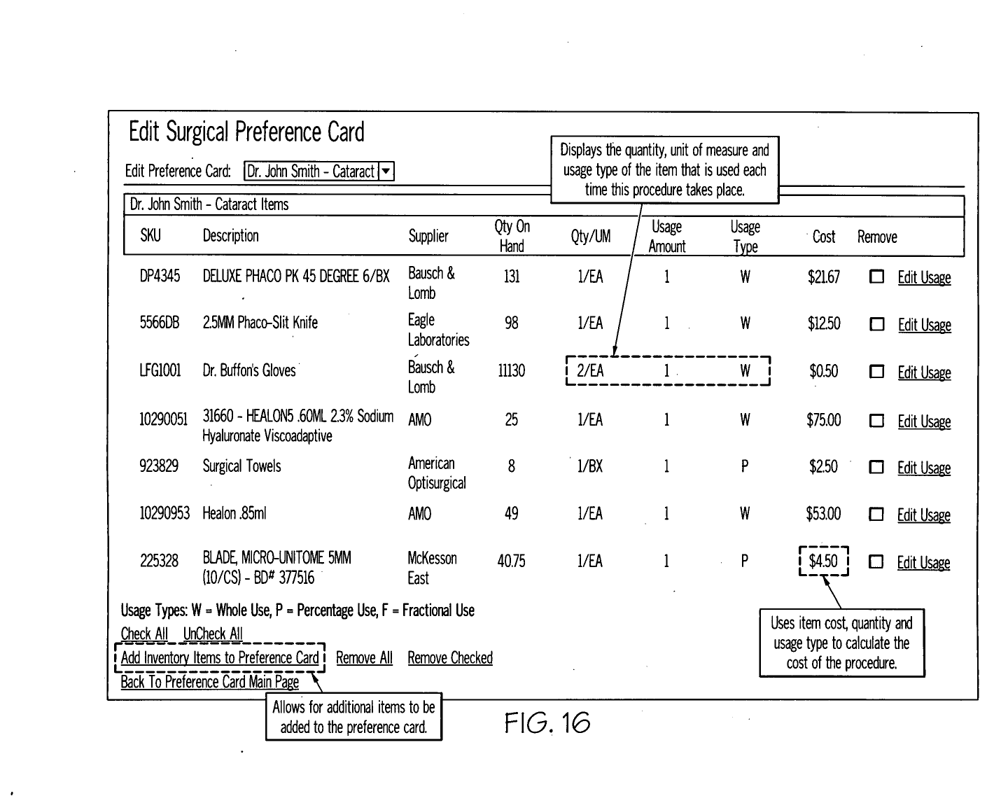

The inventoried items tracked by the system and method can be utilized by the user in various procedures, manipulations, assemblies, manufacturing, etc. Continuing with the illustrative example outlined above, a uniquely-identified implant may be surgically implanted into a patient/recipient in a surgical procedure. As part of the procedure, other items such as towels, scalpels, gloves, sutures, saline solution, gurneys, pharmaceuticals, etc. may be utilized. As shown in FIG. 16, a preference data form (also known as a preference card) may be generated by the system and method for a given procedure by a particular physician. The preference data form may list all of the items that are expected to be used in that particular surgical procedure, and list the number of uses for each item. Each physician may have his or her own preference data form for each procedure to reflect that physician's usage and preferences. For example, a particular physician may vary from other physicians in his or her use of particular items, use of quantities of items, preference of a particular brand of item, etc.

The system and method may have the capability to suggest changes and/or updates to the preference data form. For example, a preference data form for a certain physician with respect to an ocular lens replacement procedure may specify seven surgical drapes. However, the system and method may note that the physician typically uses only five surgical drapes for that procedure. In this case, the system/method may notify the user of the discrepancy and suggest a reduction in the number of surgical drapes in the preference data form. The system may also note that a particular physician consistently exceeds values specified on a data form for a given procedure, and suggest an appropriate increase for that item on the preference data form. In this manner, the system and method can suggest adjustments to the predictive data used on a data preference form, thereby minimizing costs and maximizing efficiency for the user.

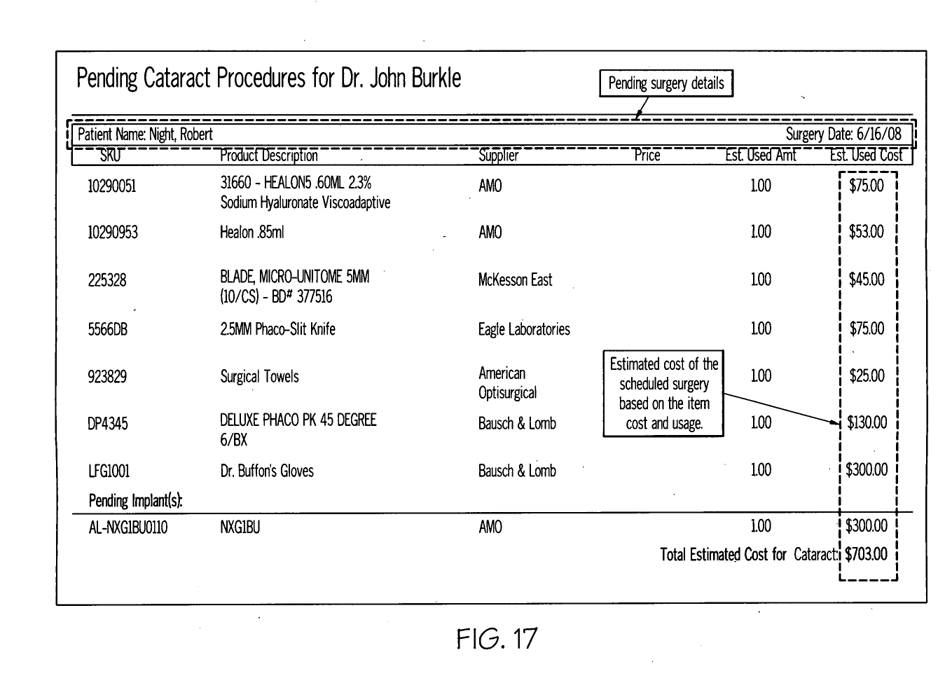

Since the system and method includes, or has access to, the user's inventory databases, predictive cost analysis can be run for each procedure based upon the preference data forms. In particular, since the preference data form lists all of the items to be utilized or consumed in a procedure, and since the inventory database provides the costs associated with the listed items, the total predictive costs for a procedure can be instantaneously generated by multiplying usages of items by the associated cost. An example of a screen shot showing total predictive costs for a procedure is shown in FIG. 17. Whole, percentage, or fractional usage of items for a give procedure can be applied to the cost for that item as appropriate. In addition, the appropriate costing information can be accessed based upon the LIFO or FIFO method dictated by the user for its inventory. Thus the system and method provide a powerful predictive tool that can generate, in real time, predictive costs for a given procedure.

b. Procedure Tracking

All the events and item usages during a procedure, or over a particular period of time, may be tracked. For example, before a procedure begins, a procedure tracking page or form, may be opened, and the identity of the patient, the identity of the physician and staff, the type of procedure, the items expected to be used (i.e. from the data preference form) may be entered. The identification of the patient can be entered by any of the means described above for inventorying items, such as manually entering identification information (i.e., via a keyboard), or scanning a bar code or an RFID tag use, voice recognition, or by the use of retinal scans, fingerprints or other biometric information. In the case of a bar code, or of an RFID tag or the like, the bar code or RFID tag may be physically associated with the patient (i.e., located on a wristband worn by the patient). Alternately, the patient identification information may be located on the patient's chart (i.e. in the form of a bar code or RFID tag), printed out and provided at various locations in a facility, or otherwise accessible by the user.

The procedure tracking function of the system and method allows the user to track particular events associated with an event, patient, or the like. For example, after the procedure tracking form is set up, the time at which the patient first enters the surgical room may be entered. This information may be entered by any of the means described above for entering patient identification information (i.e. manually, scanning a bar code, reading a RFID tag, voice recognition, etc.) In the case when a scanner is utilized, the surgical facility/user may have, or may be provided with, a bar code book or sheet that lists a plurality of scannable labels for particular events. For example, when the patient first enters the surgical room, a user may search the code book to find a bar code that is labeled “Patient Enters Surgical Room.” The user than scans that bar code with the personal electronic device, which makes the appropriate time-stamped data entry.

The user then continues to use the personal electronic device (or other data entry method) to track all pertinent events and usage of items. For example, besides noting and tracking the entry of the patient into a surgical room, various other events may be noted, as desired by the user, such as administration of anesthesia, the use of an autoclave to sterilize equipment, initial incision, irrigation, implantation of an implant, administration of saline and antibiotics, incision closure, the use of certain surgical tools and equipment, exit of the patient from the surgical room, recording required FDA or other institutional data, etc.

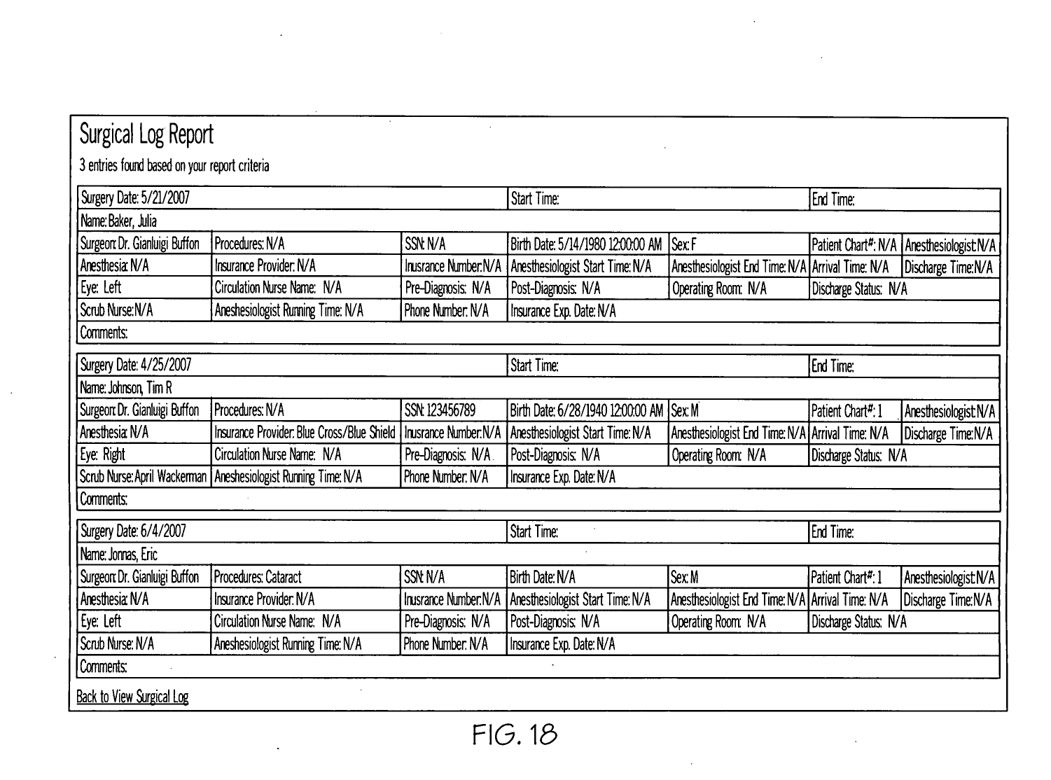

All relevant or tracked occurrences in an event, such as a surgical event, may be compiled and presented in the procedure tracking form, also termed a surgical log in the illustrative example, as shown in FIG. 18. Besides tracking specific events, elapsed time may be calculated and displayed, such at the total time required to complete a procedure or sub-procedure. Information in the surgical log can be mined and cross referenced so that the user, and/or individual physicians, can study their techniques to determine how to eliminate inefficiencies, improve techniques, etc.

The system and method may be configured to receive information directly from instruments and equipment utilized during the procedure. For example, in implanting an IOL a phacoemulsification machine may be utilized to emulsify the patient's lens for ease of removal. The phacoemulsification machine may track certain procedure and events (such as time of operation, irrigation rate and time, time required to replace a needle, etc.). The data from the phacoemulsification machine may be automatically or manually, directly or indirectly provided to the system and method (i.e., in one case, by connecting to a USB port of the phacoemulsification machine).

The procedure tracking form may also be utilized to update inventory data for the items utilized during surgery. As noted above, certain events may also be associated with usage of inventoried items. In the case of an implant procedure, the surgical log may note and track the unique identification number for the implant, so that relevant information relating to the implant can be extracted and entered into the procedure tracking form, and the inventory levels for the implant correspondingly reduced. As a further example, irrigation of the eye during surgery may count as a usage event, and thereby reducing the inventoried amount of saline. In this manner, an accurate, real-time picture of inventory is maintained.

The surgical log can be particularly useful in that it may contain information which is mandated to be tracked, such as by federal and/or state regulations, as well as information requested to be tracked by the user, so that the relevant information is collected into a single log. For example, a surgical log may track implant details and anesthesiologist running time, which can aid in tracking efficiency, generating bills for an anesthesiologist who bills by the minute, etc. If desired, the system may provide restricted access to the surgical log reports such that only a physician or other authorized user has the ability to review surgical log reports.

Moreover, various pre-surgical and post-surgical events may be tracked and incorporated into the tied to a particular patient record. For example, admission of a patient, pre-surgical counselling, signing of a consent and release, etc., may be tracked. Post-surgery events, such as discharge time, time of post-surgical counselling, administration of medication, recovery thresholds etc., may also be tracked. Moreover, the post-surgical events may also extend to cover return visits by the patient, short and long-term surgical outcomes, etc. In this manner, the procedure tracking form provides complete tracking of events and item usages. In addition, a user's electronic medical records may be automatically updated to incorporate the procedures noted in the surgical log.

The surgical log is an example of the benefits offered by the data aggregation provided by the system and method. In particular, disparate data is created in various forms and locations by various entities, such as, by way of example, during the admission and discharge of a patient, inventorying of items, placing and receiving orders from a supplier, storage of data on a web server, utilizing an implant, etc. As can be seen in the second surgical log entry of FIG. 18 (having a surgery date of May 23, 2007), the start and end time for that procedure are indicated, as provided by data entry, possibly by scanning a barcode or RFID tag during the surgical event. The model, diopter, and serial number of the implant is listed. This information may have been provided, for example, from the supplier several weeks before actual usage. Information relating to the surgeon name and operating room may also be collected; this information may have been created weeks or months prior when the surgery was scheduled. A label may be provided on the saline which identifies the saline in a manner desired by the surgeon, which label may have been created at the time of inventorying that product.

Thus it can be seen that various data (often provided in small bits) is entered and tracked at various locations by various personnel for disparate purposes. The system and method collects the disparate forms of data, organizes the data, and provides useful results from data which otherwise might not be tracked or utilized, or used together in the manner shown herein. The various types of information thus have multiple uses, and can be used together in varying combinations to suit the needs of the user.

c. Data Preference Form Variances

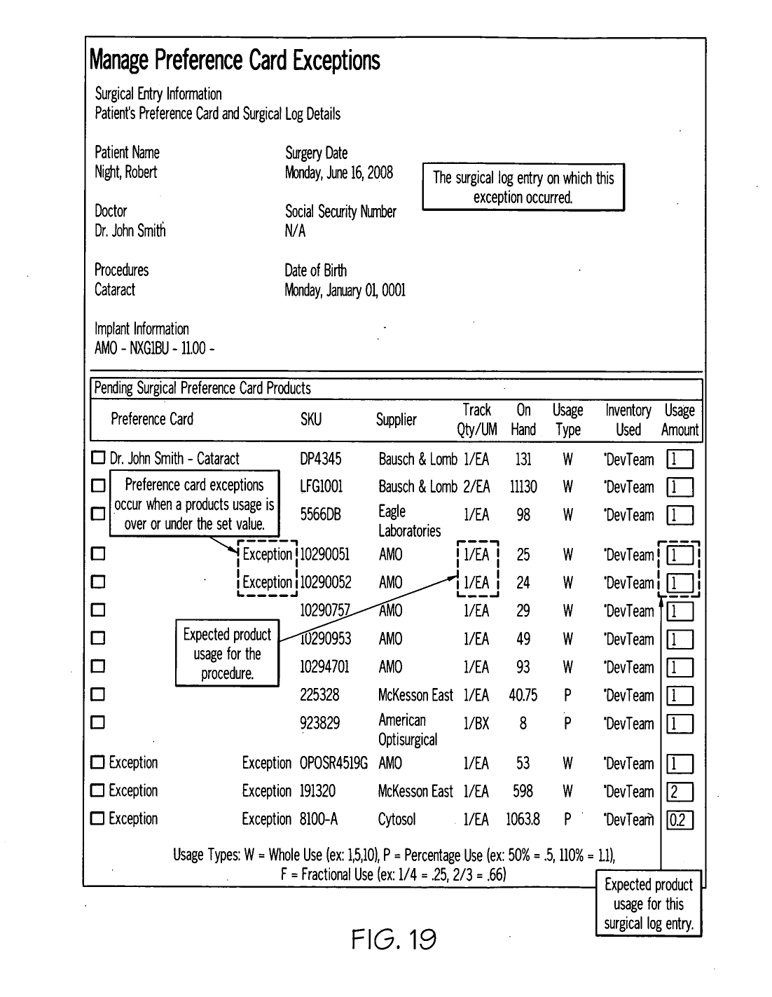

As a surgical event or other procedure progresses, the actual usage of items may vary from that listed on the preference data form. For example, in one case the data preference form may predict the use of a single scalpel, but the actual procedure may require the use of two scalpels. In this case, the variance may be termed a preference data form “exception,” and the preference data form may be modified in real-time to account for this variance. FIG. 19 provides an example in which preference data form exceptions are managed and entered into the system. In the example shown therein, two usages of two different products were implemented, when only a single use of each product was predicted.

The entering and tracking of exceptions allows the user's inventory to be maintained at accurate and up-to-date levels, and allows for accurate case costing. In addition, the tracking of exceptions allows the data preference form to be presented as a staring point, and variances entered as they occur, such that actual usages can be accurately tracked without having to enter each and every usage. If desired, variances to the data preference form may be entered automatically if detected from data entries made in the procedure tracking form.

After all of the exceptions have been entered to a preference data form, the resultant form provides actual usage data relating to that procedure. After sufficient data has been gathered, the system and method may note common exceptions to the preference data form, and provide a suggestion to the user to modify the preference data form. Common exceptions may also be noted and changed directly by the user without prompting from the system. In this manner, the various field on the data preference form are constantly analyzed and updated (or suggested to be updated) to ensure that all of the necessary items are provided, and extraneous items excluded from the preference data form, to increase the efficiency and reduce costs.

In addition, or alternately, common variation or exceptions may be noted by the system and/or the user prior to a surgical event (i.e., at the time a surgery is scheduled). At that time, inventory levels may be checked, and/or the associated items may be accessed, to ensure that the common variations or exceptions are ready and available, even if they are not actually incorporated into the data preference form. For example, if a common exception for a particular procedure is the use of one extra scalpel, the system may cue the user, and/or automatically check inventory, to ensure that an extra scalpel is on-hand and/or available for possible use. In this manner, the exception management feature of the system helps the user to anticipate, and prepare for, common variations such that when such variations arise they are easily accommodated. Additionally, common variations and exceptions can be provided to the user to allow easy data entry for the most common exceptions.

d. Automated Forms Management

When a surgical event is complete, a “bill and replace” event may be automatically generated by the system and method. In particular, when an item (such as an implant) is utilized in an intraocular lens replacement procedure, the user may be required to notify the supplier (particularly if the implant was on consignment from the supplier). In this case, relevant information relating to the procedure, such as the unique identification number of the implant, identity of the patient, date of procedure, name of surgeon, name of the surgical facility, etc., may be identified and/or extracted by the system. The relevant information is then automatically or semi-automatically (i.e., automatically with approval, or aggregated with other information for that supplier) sent to the supplier by computer communications, fax, email, EDI or otherwise, so that the supplier can generate an invoice or billing information. Once the supplier receives the order and/or notification, replacement products may be shipped as desired. However, certain products that are not part of a consignment arrangement (i.e. implants purchased directly from a supplier or the supplier's agent) would not necessarily be replaced upon use.

Additionally, FDA or other regulatory or government programs may require that relevant information about the implant and/or procedure be reported to the FDA. The FDA-required information may be the same as, or different from, the information required to be sent to the supplier. The system and method has the ability to compile the particular information required by the FDA, present the information in a format acceptable to the FDA, and send the information to the FDA by computer communications, fax, email, EDI, or otherwise. In this manner the user can comply with FDA or other regulations relating to human medical implants with minimal effort on the user's behalf The user, host and/or supplier may send the appropriate information to the FDA once the user indicates that the procedure/patient is “complete.”

Since each surgical procedure requires a “bill and replace” event and FDA reporting, the system and method disclosed herein provides a automated series of events triggered by end use of an implant in which “bill and replace” and FDA notification are carried out automatically or semi-automatically, thereby greatly reducing workload upon the user.

e. Expense Tracking

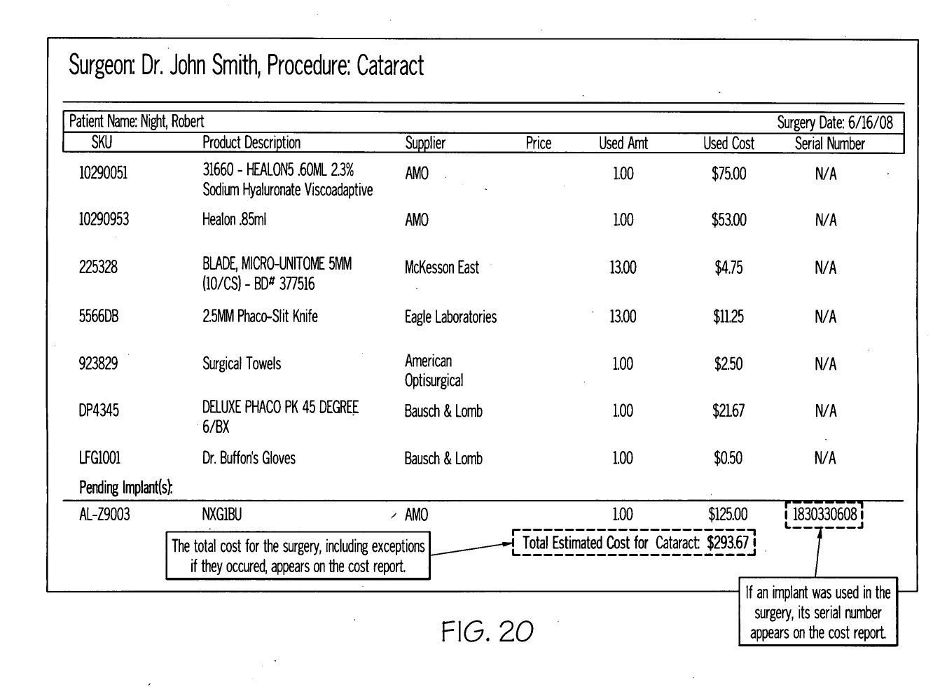

The system and method may be utilized to track actual expenses incurred in a particular event, an example of which is shown in FIG. 20. In particular, since the system tracks all of the items utilized or consumed in a procedure (in particular, by the data preference form, with any exceptions entered therein), and since the inventory database provides the costs associated with the listed items, the total costs for a procedure can be instantaneously generated, as shown in FIG. 20. The data can be generated from a data preference form that has been modified with variances, or from a procedure tracking form, or from combinations of the two. Whole, percentage, or fractional usage of items for a given procedure can be applied to the cost for that item as appropriate, using appropriate LIFO/FIFO accounting as dictated by the user during item intake/inventorying.

If desired, the actual total costs may be compared to the predictive costs for a procedure (an example of which is shown in FIG. 17). Moreover, the actual costs can be uploaded into the user's accounting software to allow seamless transition of data for accounting purposes. In addition, such case costing information can be analyzed to allow the average cost of certain procedures to be tracked, to track certain cost trends, identify particularly expensive or inexpensive procedures, identify certain physicians or items which positively or negatively effect costs, etc. to provide a powerful efficiency-measuring tool.

In this manner, the system provides the underlying logic to track, maintain, update, use, receive, reorder and report on the entire lift cycle of item and procedures. Data from suppliers, surgical log data, physician preference data, implant data, and inventory details are also integrated into the system to provide a seamless and wide-ranging system to track and integrate disparate types of information into a single system.

7. Facility Management

The system and method disclosed herein may also be utilized to track, reserve, and manage facilities. The facilities may be used in association with the items and procedures which are tracked in the manner described above, although the facilities need not necessarily be used with the tracked items. For example, continuing with the illustrative example, the facilities managed in this case may constitute surgical/operating rooms in which inventoried implants are utilized.

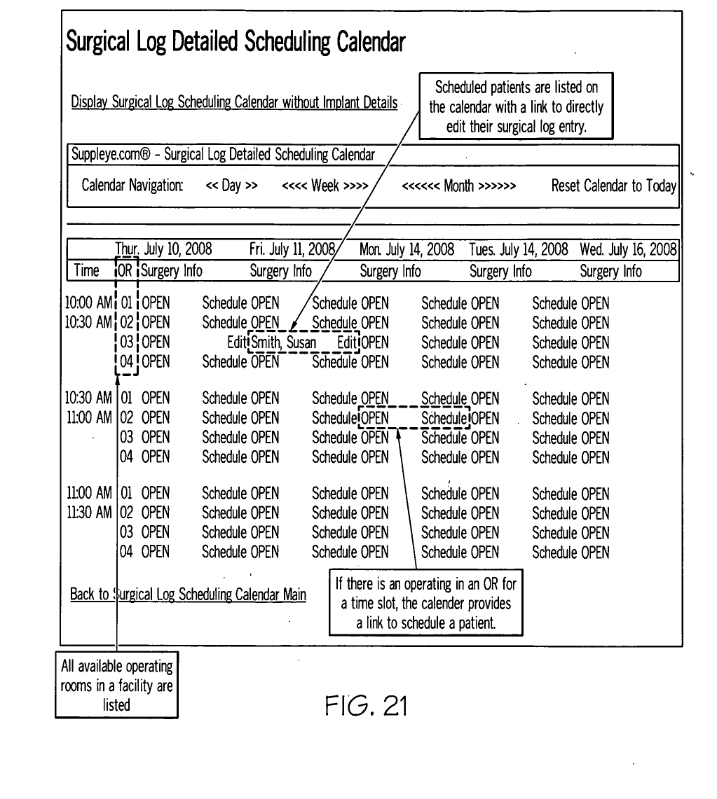

As shown in FIG. 21, the facility management system and method may take the form of a calendar-based display which conveys information about particular facilities for a given day. For example, the calendar shown in FIG. 21 displays the status for four separate surgical rooms (Rooms 01-04), between 10am and 1pm, for Jul. 10, 2008 and Jul. 14-Jul. 16, 2008. Differing days and times can be viewed by clicking on the calendar navigation buttons at the top of the screen.

Each room has a status indicator for each hour of each day (i.e. either open or scheduled/reserved). If a room is scheduled for a particular time, certain information (such as the name of the patient, the name of the associated physician, the type of procedure, the implants to be utilized) or other indicia (i.e. the text “reserved” or the like) may be displayed (either directly, or as a pop-up when the display is rolled over the by mouse pointer, etc.) to signal that the room is scheduled. Further information about the scheduled procedure can be displayed by clicking on the link indicating that the room is reserved. For example, the name of the patient, the name of the physician, the type of procedure, implants to be utilized, the preference data form, etc. may be viewed, and/or edited, in a separate screen or window that is opened when the associated link is clicked.

A room that is indicated to be open or unscheduled can be reserved by clicking on the associated link. All of the relevant information for the procedure to be scheduled can be entered in a form presented in a new window or screen. Once the relevant information is entered into the reservation form, the information is stored and the status indicator for the room is changed to “reserved” for the appropriate time period.

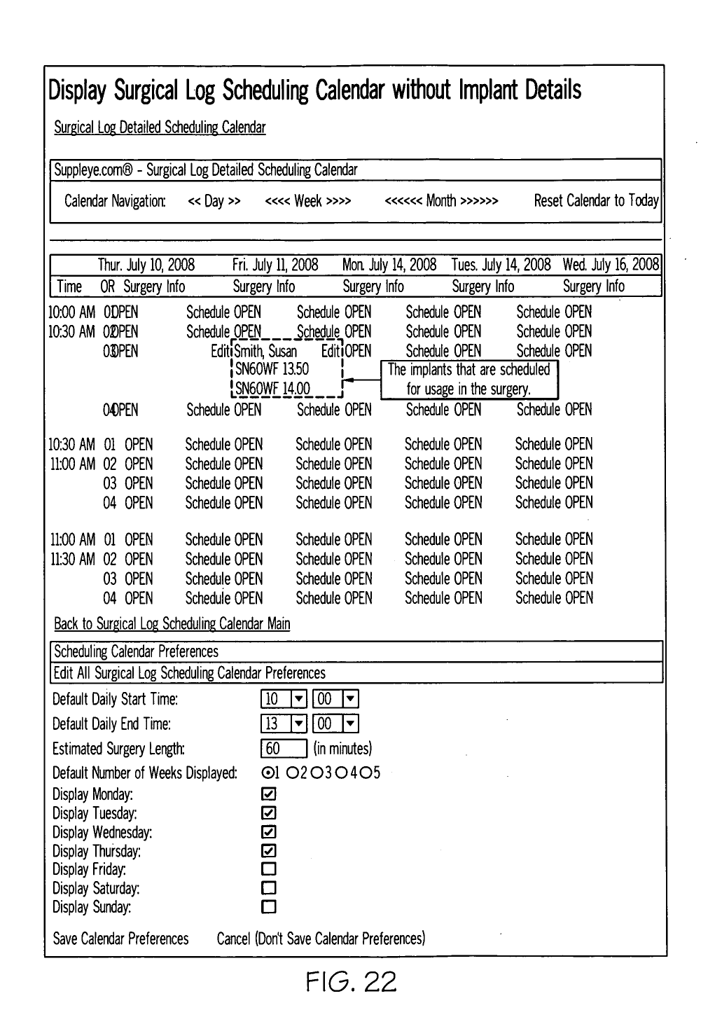

As shown in FIG. 22, a scheduling calendar preference module may be provided to aid the user in identifying available facilities meeting certain criteria. For example, the user may enter the surgical start and end time (which may include preparation and post-operative procedures). The days of the week which are desired to be displayed and the number of weeks to be displayed in the search results are then selected.

This system and method for scheduling procedures allows users to schedule procedures in an intuitive manner by the user's computer via the internet, or by a user's personal electronic device from a remote location, etc. The scheduling system provides a visual, user-friendly representation of scheduled events and allows the user to check and modify information and schedules as desired. Moreover, the scheduling system can be linked to and/or synchronized with other software systems, such as calendar and/or email systems (i.e. the MICROSOFT® OUTLOOK® system) such that notification and alerts to the participants in a various procedure are automatically provided.

When a procedure is scheduled, the user's inventory may be correspondingly adjusted. For example, when a physician schedules a particular procedure, the preference card for that particular procedure for the scheduling surgeon may be automatically accessed. Since the preference card provides a prediction of the uses of inventories items, inventory levels can be appropriately reduced, or projected to be reduced.

The user of the system may provide various levels of access to the scheduling calendar for differing ones of its employees, contractors, agents, etc. For example, in the illustrative example, if a user is designated or identified as a “physician” then that user may have a greater ability to access and modify the scheduled as compared to non-physician users. For example, only a physician user may be permitted to schedule, view and edit view history reports associated with their own surgeries. In addition, only physician users may be able to access the calendar screens/methods described above and shown in FIGS. 21 and 22. The facility management system/scheduling system thus allows users to avoid scheduling conflicts and inventory shortfalls.

Users of the system and method used herein may utilize other tracking software and system which may be desired to interact with the system and method described herein. In particular, in the case of the illustrative example, a user may utilize electronic medical records (“EMR”) software system, which is typically associated with the user's accounting software system. The system and method of the present invention may be able to detect and determine whether the user utilizes EMR software, and provide data compatible with that system, and/or communicate directly with the EMR system, using artificially intelligent logic. For example, the system and method disclosed herein may import relevant data, such as data related to patients for a scheduled surgery, into its databases from the EMR software. Thus the present system reduces duplicate data entries and minimizes errors due to the ability to automatically import data.

The EMR software/databases to be used by the system may be selected simply by providing a “browse” EMR button which the user can select to navigate its computer's hierarchy to select the appropriate software/database. The artificial intelligence of the system and method may minimize the time and input required by the user. However, certain features relating to the data imported from the EMR software may be controlled and customized by the user, such as what sort of data is imported, how long the imported data is maintained, what sort of access is provided to the data, etc. In a similar manner, a conduit established between the present system and method and the EMR software allows export of surgical details directly to the EMR database, such as the identifier of the implant, details of the surgical procedure, etc., to maintain accurate patient records, thereby improving overall accuracy while reducing clerical burdens.

8. Implementation

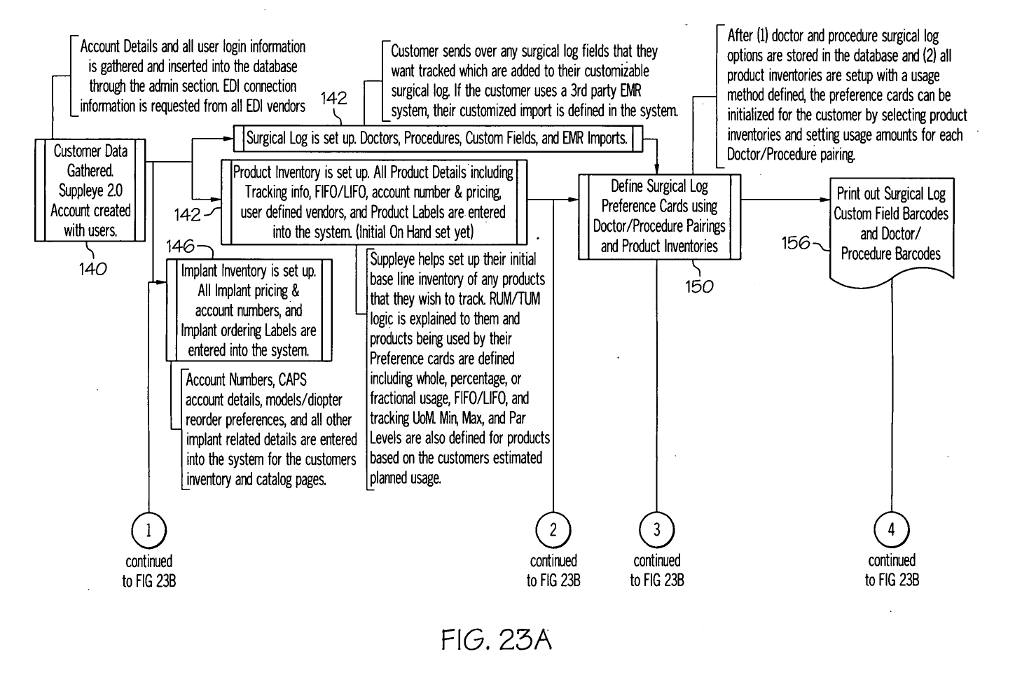

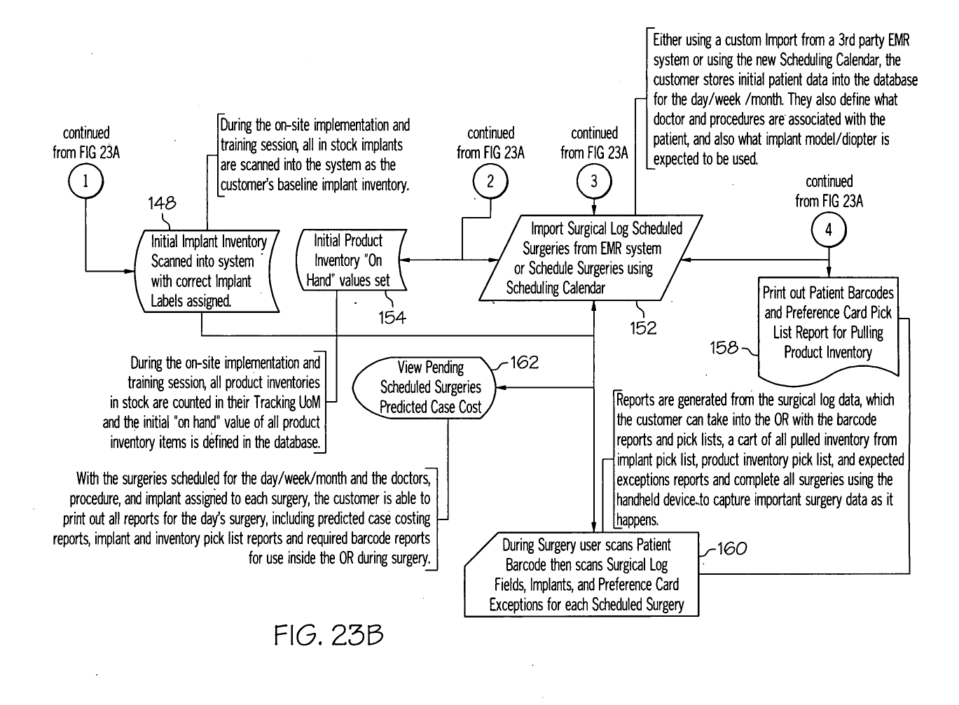

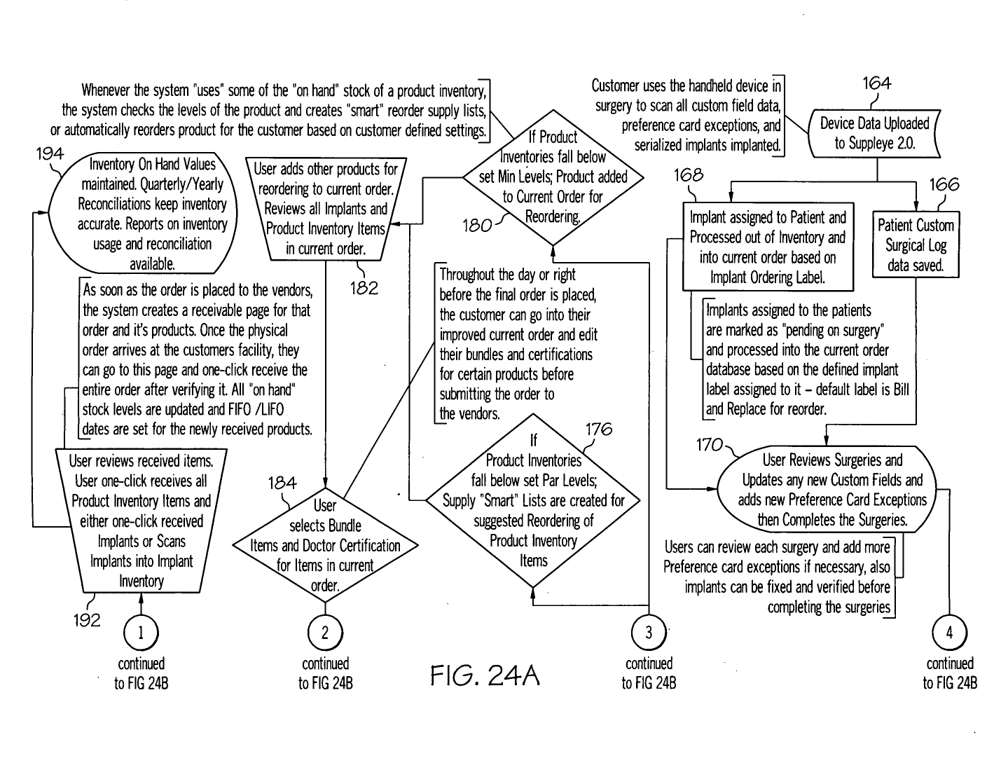

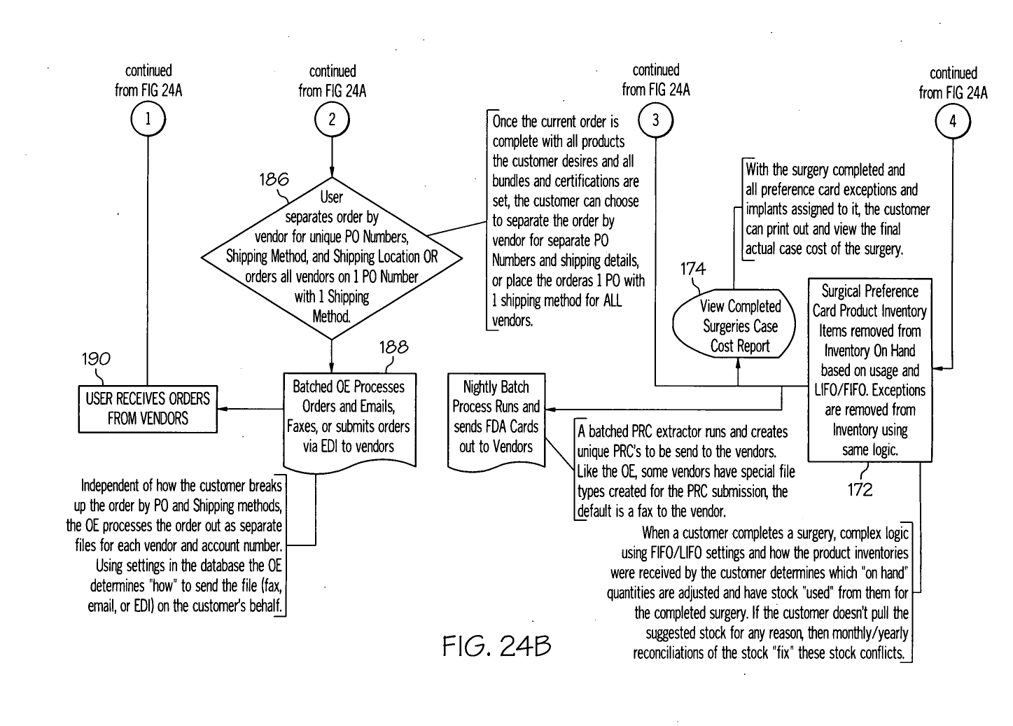

FIG. 23 is a flow chart, with various annotating comments, illustrating how a new user can be integrated into the system and method described herein, and make initial use of the system and method. The various steps in FIG. 23 are described in a particular order out of convenience. However, it should be understood that the various steps of FIG. 23 can be carried out in nearly any logical order, and more particularly in the various orders as identified by the connections between the various steps shown in FIG. 23. The same also applies to FIGS. 24-27 described below.

With reference to FIG. 23, at block 140 the customer data is gathered, and an account is created for that user. Account details and user login information are added to a database and, if appropriate, EDI connection information or other communication link information between the user, host/operator and any suppliers is exchanged. At step 142, details relating to a procedure tracking form/surgical log for that particular customer are inputted. In particular, the customer communicates the fields/information which they would like to have tracked in their surgical log. If the customer uses an EMR system, the manner in which EMR data is imported and treated by the system/method may also be specified at this time. At step 144, the item inventory is defined/set up as described in Section 3 above. For example, item details including item identification, tracking information, accounting tracking designations (i.e., FIFO/LIFO designations) account numbers and pricing information for particular suppliers, item labels, etc., are entered. Moreover, initial baseline levels of inventory, minimum, maximum and par values for each item, usage type indicators (i.e., whole, percentage, or fractional), reorder unit and tracking unit measure (RUM/TUM) logic (defined above), etc., can be set up at this time.

At step 146, the user's inventory of items is set up, including pricing and account numbers, particular account details (i.e., whether a CAPS account exists for particular items), reorder preferences, and other details. At step 148, the initial or on-hand inventory of implants or other uniquely-identified items may be entered into the system. Next, at step 150, the preference cards/preference data forms can be set up for the particular preference for each physician of the user. Defining the preference card/preference data form takes into account the accounting tracking designation and usage type of the various items as defined, for example, in step 144.

At step 152, the surgical log, scheduled procedures and associated data from the user's EMR system may be imported or uploaded into the system and method. The uploaded/imported data also defines a relationship between each physician and associated procedures and patient, as well as what particular implant is expected to be used. This will allow the system and method to accommodate existing scheduled procedures when scheduling new procedures, surgeries or procedures using a scheduling calendar. At step 154, the initial item inventory, or “on hand” values of the user are entered into the system. These “on hand” values may be counted or expressed in their tracking units of measure, which may be more meaningful to the user than other units of measure.

At step 156, the surgical log/procedure tracking form barcodes and doctor/procedure barcodes (i.e., for use in creating data to fill the surgical log) may be printed out for use in a particular procedure. At step 158, patient barcodes (i.e., patient identifiers) and a preference card pick list may be printed out to aid the user in pulling the items in preparation for a particular procedure.

During a subsequent surgical event, the user can then scan the patient barcodes and surgical log fields (i.e., printed out in steps 156 and 158) to populate the surgical log fields, as noted at step 160. Exceptions to the preference cards can also be entered by scanning barcodes or, as noted above, various other forms of data entry. This allows the user to generate reports from the surgical log data and capture surgical data in real time or near real time. Finally, at step 162, the user can view pending, scheduled surgeries or events, as well as predicted case costs associated with each scheduled surgery.

FIG. 24 is a flow chart, with various annotating comments, illustrating various steps involving the use, tracking, and reordering of items under one embodiment of the current system and method. In particular, beginning with step 164, the data captured during a surgical event (i.e., via a personal electronic device) can be uploaded to the system and method. In this manner, all of the data captured with a hand-held device during surgery, including preference card exceptions and identification of items utilized during surgery can be accurately tracked. At step 166, information which the customer has specified is to be tracked in its surgical logs (i.e., in step 142 of FIG. 23) is saved, although all tracked data can also be saved at this time. At step 168, an implant in a patient is then assigned to, or associated with, that patient, and inventory is correspondingly reduced. If the implant is indicated by inventory or otherwise to be a consigned item, a “bill and replace” event may be automatically generated.

At step 170, the user can review information generated during the surgical events, such as a surgical log, update new custom fields which may be desired to be tracked, and enter any exceptions to the preference cards (which exceptions may be suggested by the system and method, or which may be initiated by the user). Step 170 also allows the user to verify use of the correct implant.

Next, at step 172, items indicated to be used in the surgery, such as those listed on the preference card, are removed from the “inventory on hand” levels maintained by the system and method. The accounting tracking designation and usage type of the item is taken into consideration during this step. Moreover, any exceptions made to the preference card are taken into account (i.e., use of additional items are reduced from inventory and non-use of items is also noted). At step 174, the user can view the completed case cost report for that surgery. This provides a user with an accurate real-time or near real-time picture of the actual cost of all materials consumed in a procedure.

At step 176, if the reductions in inventory (i.e., carried out at step 172) reduces the inventory for that item below par level, the system and method suggests reordering to replenish inventory levels. Similar steps are triggered at step 178 if inventory levels fall below the minimum level. In either case, if inventory levels fall too low, the user may be directed to an order form/reorder form with a virtual shopping cart which identifies items desired to be acquired by the user. The item which falls below par or minimum levels may be automatically added to the shopping cart in sufficient quantities to replenish to par levels or otherwise. In this case, as at step 182, the user can also add other items to the shopping cart/order form/reorder form. Information relating to the inventory levels for each item indicated to be ordered (i.e., in the shopping cart) may be provided to aid the user in determining quantities to be ordered. At step 184, if appropriate, bundled items and/or certification may be required in order to meet requirements for that particular order/supplier, as described above.

At step 186, it is noted that the system and method may separate or segregate the order, or otherwise indicate to the user the identity of differing suppliers, items under unique purchase order numbers, or the use of shipping methods and/or shipping locations for various items within an order, as described above in Section 4.b. This allows the user to customize the order for differing suppliers and shipping methods as desired.

At step 188, the system and method receives the order information supplied by the user and extracts, organizes and groups the various orders in a batch process as appropriate. As noted above in Section 4.d., the system and method may maintain a supplier database which stores the requirements or preferences of each supplier. The system and method can then place the information received from the user in a format preferred or accepted by the supplier and forward the information in the desired manner (or cue the user for additional information, if required). Thus, at step 188, a single order submitted by the user can be processed as multiple orders for each supplier and account number. The order information can be forwarded to the supplier in a variety of manners depending upon the supplier's requirements or preferences. At this time, the system and method may also create a receivable page for each order and its associated items, which stores the relevant information in the order such as, for example, item identification, manufacturer, quantity, etc.

Once the supplier has received the order from the host/operator, identified the appropriate items and shipped them to the user, the user then receives the order of items as indicated at step 190. Once the user receives the items, the receivable page (created at step 188) for that order can be accessed by the user (i.e., by accessing, via the internet, a database associated with the system and method). The user can then navigate to the appropriate page and receive the item into its inventory by manipulating the receivable page, as noted at step 192 and described in Section 5.a. above. As noted at step 194, the user then continues to maintain and update its on-hand values and can conduct periodic reconciliations to maintain accurate levels of inventory.

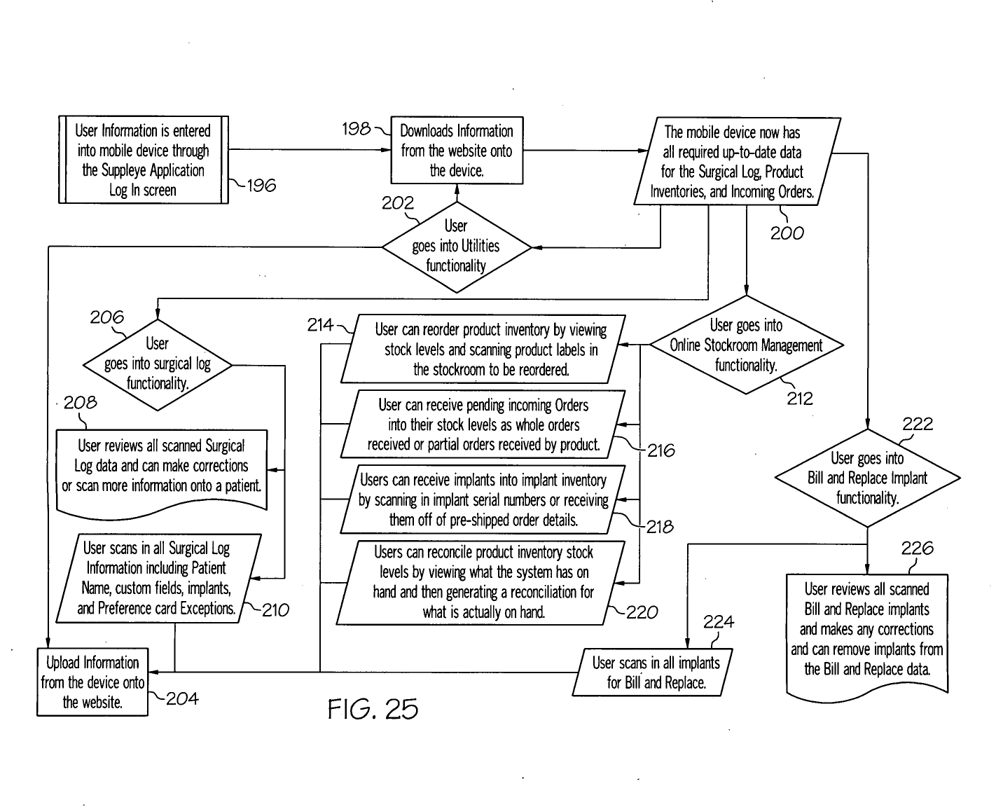

FIG. 25 is a flow chart illustrating various uses and functionality of a personal electronic device in the system and method disclosed herein. Beginning at step 196, user information can be entered into the mobile device/personal electronic device such as by a cable, docking system, hot-sync system, wireless connection, etc. The user may be first required to log in to the system and method in the same manner as noted above for logging in using the user's computer or the host computer. Next, at step 198, information is downloaded from the host onto the personal electronic device. The download may include relevant information for operating the personal electronic device and system/method, including identity of the user, preference card data, accounting tracking designations, usage types of items, surgical logs, item identifiers, item inventories, status of incoming orders (i.e., receivable pages), as well as nearly any information stored on the user's computer, host computer and/or required by the user to utilize the system and method. As shown at step 200, after the download is complete, the personal electronic device is ready for full use.

As shown at step 202, the personal electronic device may be able to be used in a utilities function in which various user preferences, screen displays, power-on and power-off settings, communication protocols and the like can be controlled. The changes can then be uploaded to the user's computer and/or the host computer at step 204.

At step 206, the personal electronic device can be used to provide surgical log functionality. In this case, as described above, surgical log data can be entered (such as by scanning a barcode) during a surgical event, in real-time or near real-time using the personal electronic device. At step 208, the user can review the scanned surgical log data and, at step 210, the surgical log information including, for example, patient name, custom fields, implants and any preference card exceptions, is scanned. The data can then be uploaded to the user's computer and/or the host computer at step 204.

As noted at step 212, the personal electronic device can be utilized to provide stockroom management functionality. For example, at step 214, a user can utilize the personal electronic device to reorder items by viewing stock levels on the personal electronic device, and scanning item labels (i.e., barcodes) to identify an item for reorder. Pertinent details relating to the item (i.e., current stock levels) can then be retrieved by the personal electronic device. The user can then enter information relating to the order/reorder (i.e., quantity, identity of the supplier, etc.) via the personal electronic device.

As shown at step 216, pending incoming orders can be received and uploaded into the inventory database by scanning one or more barcodes or otherwise entering information relating to the received order. The receivable page can then be manipulated as desired, as described in Section 5.a. above to receive the order, or part thereof, into the system and method. Alternately, as shown in step 218, individual items can be received into inventory by scanning their unique identifiers or entering other relevant information by the personal electronic device. As shown in step 220, the personal electronic device can be utilized to reconcile inventory levels by viewing on hand inventory levels on the personal electronic device and then entering a reconciliation based upon actual item count as noted in Section 6.d. above.

As shown in step 222, a user can also use the personal electronic device to provide bill and replace functionality. As noted at step 224, a user can scan in an implant that is being used to trigger a bill and replace event, as noted in Section 6.d. above. Next, at step 226, the user can review all the scanned bill and replace implants, make any corrections, and remove implants, as desired, from the bill and replace data.

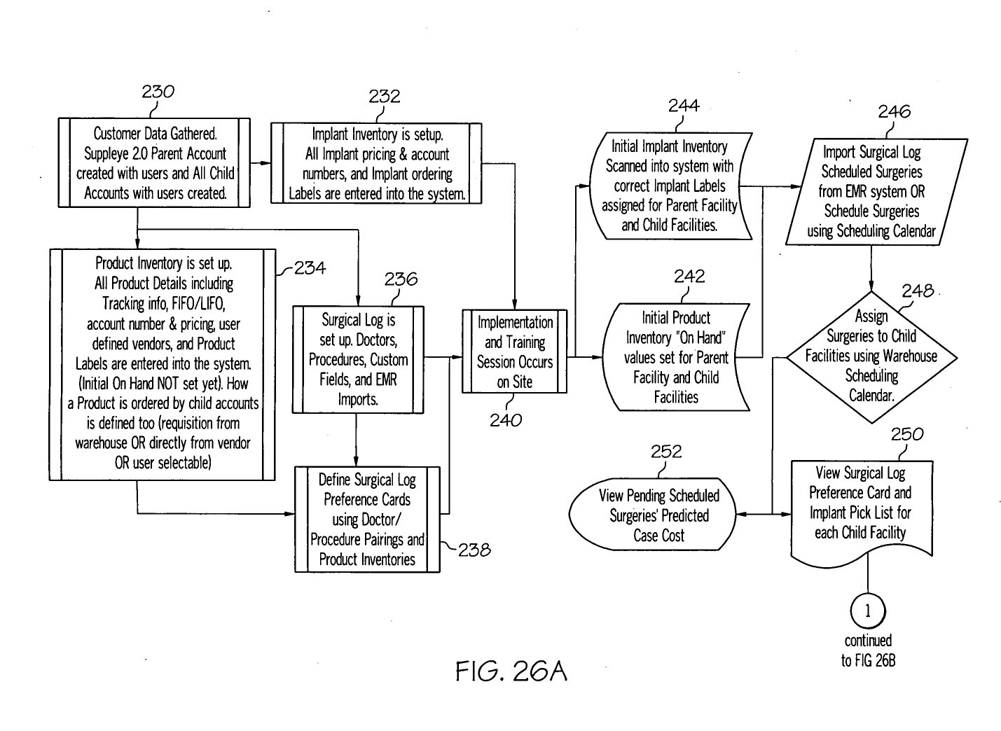

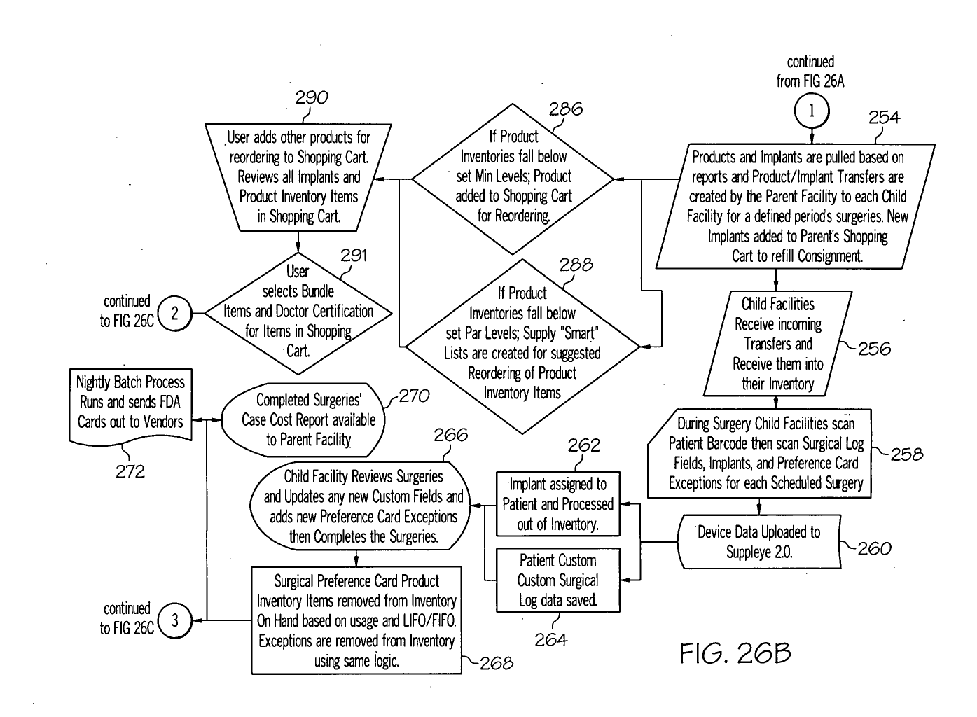

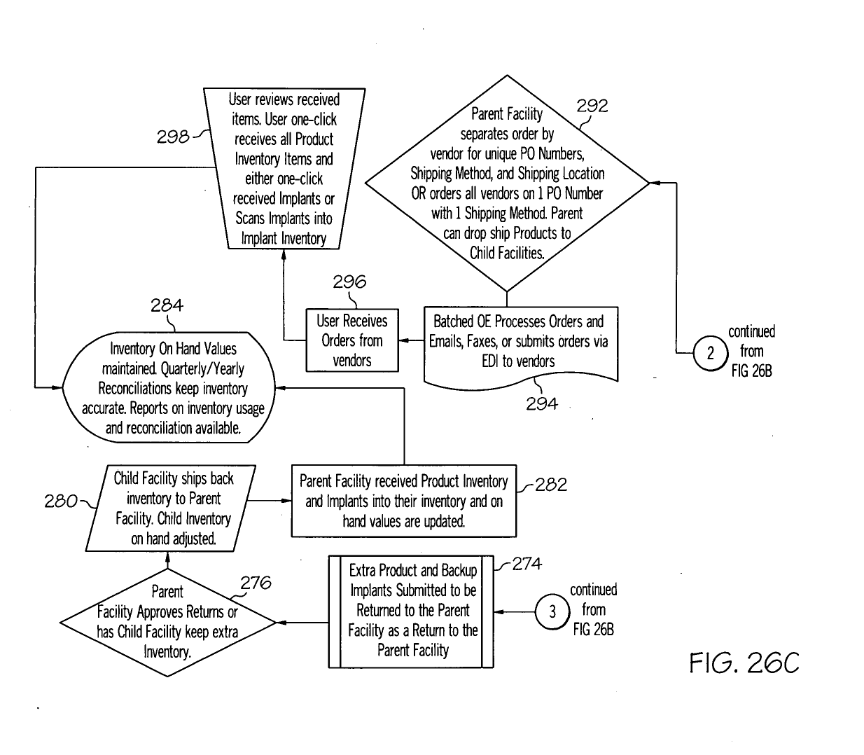

FIG. 26 is a flow chart illustrating implementation and operation of the system and method, with further particular details relating to supplier and user relationships (i.e., parent/child relationships). Beginning at step 230, customer data is gathered, and corporate or affiliate relationships are noted, such as parent/child relationships between users, suppliers, and between multiple suppliers, as noted above in Section 4.c. At step 232, the implant inventory (i.e., inventory for uniquely-identified items) is set up, including pricing and account information, as well as other relevant information. Ordering labels may also be entered into the system/method at this time. At step 234, the non-implant item inventory is set up, including inventory details such as tracking information, accounting tracking designations, account numbers, labels, pricing information, and the identity of suppliers and products. In addition, at step 234, users can define the manner in which the product is ordered. For example, if the user has a child/parent relationship with the supplier, at step 234, it is determined whether the user can receive the item by requisition from a warehouse, or directly from a supplier/vendor, or whether parent consent is required.

At step 236, the surgical log is set up in the same manner as in step 142 of FIG. 23. At step 238, the preference cards are defined in the same manner as in step 150 of FIG. 23. Next, at step 240, the system and method is installed/implemented and the user is trained in using the system/method, typically on-site at the user's facilities. At step 242, the initial product inventory “on hand” values for both the parent facility and the child facility are entered. At step 244, the initial implant inventory (or other uniquely-identified items) are scanned or otherwise entered into the system. The inventory is assigned to the parent facility, child facility, or other related or affiliated entities as desired. At step 246, the surgical log and other information are imported in the same manner as in step 152 of FIG. 23 described above. At step 248, procedures and surgeries are scheduled with, and assigned to, the user (child facilities in this case) using the scheduling calendar as described in Section 7 above. At steps 250 and 252, it is noted that the preference cards for physicians/procedures and associated pick lists can be viewed for each child facility, along with the predicted case costs for each scheduled procedure/surgery.

At step 254, in preparation of a surgery/procedure, items that are scheduled to be used in the procedure are pulled based upon reports and other information. If needed items (such as implants) are located at a parent facility, a product/implant transfer request form is created. This form specifies or requests that a particular implant is being transferred from a parent facility to a particular child facility for use in a particular procedure/surgery. In this case, the parent may be directed to an order form/reorder form to replenish the inventory lost by the parent. New items to replenish inventory may be automatically added to the shopping cart of the order/reorder form.

At step 256, the child facility receives the incoming transferred items from the parent and receives them into its inventory. At step 258, as items in inventory are used in procedures/surgeries, the child facilities scan the patient barcode or other patient identification to associate the steps/items with a particular patient. The desired surgical log fields, implant/item identifiers and preference card exceptions for the surgery are then scanned/entered into the system, similar to step 160 of FIG. 23 described above. Once the relevant information relating to items, procedures, exceptions and the like have been captured, the data is uploaded to the host/user, as noted at step 260.

Next, at step 262, items and implants are assigned to a patient and processed out of inventory. For example, in the case of an uniquely-identified implant, the unique identifier is associated with the patient identifier, thereby linking or associating the two. Noting usage of the implant in this case can also serve to remove it from the user's inventory. At step 264, the surgical log data relating to that particular patient for his or her surgical is stored. Next, at step 266, the child facility reviews the surgeries, updates custom fields, adds any new preference card exceptions, and notes that the surgery is complete, similar to step 170 of FIG. 24 described above. Next, at step 268, the items listed on surgical preference card which are used are removed from inventory, similar to step 172 of FIG. 24 described above. At step 270, the case costs for the completed surgeries are calculated as described above in Section 6.e. Reports can be automatically generated and sent to the user/child, parent facility, physician, or others. At step 272, the system may be set up to perform a nightly batch process run and send the resultant data (i.e., usage information of implants) in the form of FDA cards to suppliers for the suppliers' own use (i.e., for bill and replace) or for forwarding to the FDA or other regulatory agencies for reporting purposes.

At step 274, extra items that are not required for use by the child facility may be desired to be returned to the parent facility. As noted at step 276, the parent facility may approve the return of an item to the parent, or may determine that the child facility should keep the item (i.e., to reduce shipping costs if it is determined that that child user will use the item in the near future, etc.). If the return of the item to the parent is approved, the child facility then ships the item back to the parent facility. Child and parent inventory on-hand levels are adjusted accordingly, as shown in steps 280 and 282. As noted at step 284, the parent can maintain its on-hand values on an ongoing basis and make quarterly and yearly reconciliations analogous to step 174 of FIG. 24 and as described above in Section 5.a.

Backing up to step 254, after items have been pulled from inventory for use in procedures, the system and method check to determine whether the product inventory has fallen below minimum (step 286) or par (step 288) levels, and, if so, the user may be directed to a shopping cart or an order/reorder form. Supply lists may then be created/suggested, and the shopping cart may be initially populated analogous to steps 176 and 180 of FIG. 24. Next, at step 290, the user adds other products to the shopping cart in the same manner as at step 182 of FIG. 24. At step 291, the user can select bundled items and carry out steps necessary to meet certification requirements as specified by the system/supplier, and described in Section 4.b. above.

Next, at step 292, the parent facility can separate the order for display, analogous to step 186 of FIG. 24. At step 294, the batched orders are forwarded to the supplier, analogous to step 188 of FIG. 24. At step 296, the user (in this case, either the child or parent facility) receives the order from the supplier and, at step 298, reviews and receives the items in the same manner as described in step 192 of FIG. 24 (Step 298). Finally, the system and method proceed to step 284 in which inventory on-hand values are maintained and reconciliations are periodically carried out.

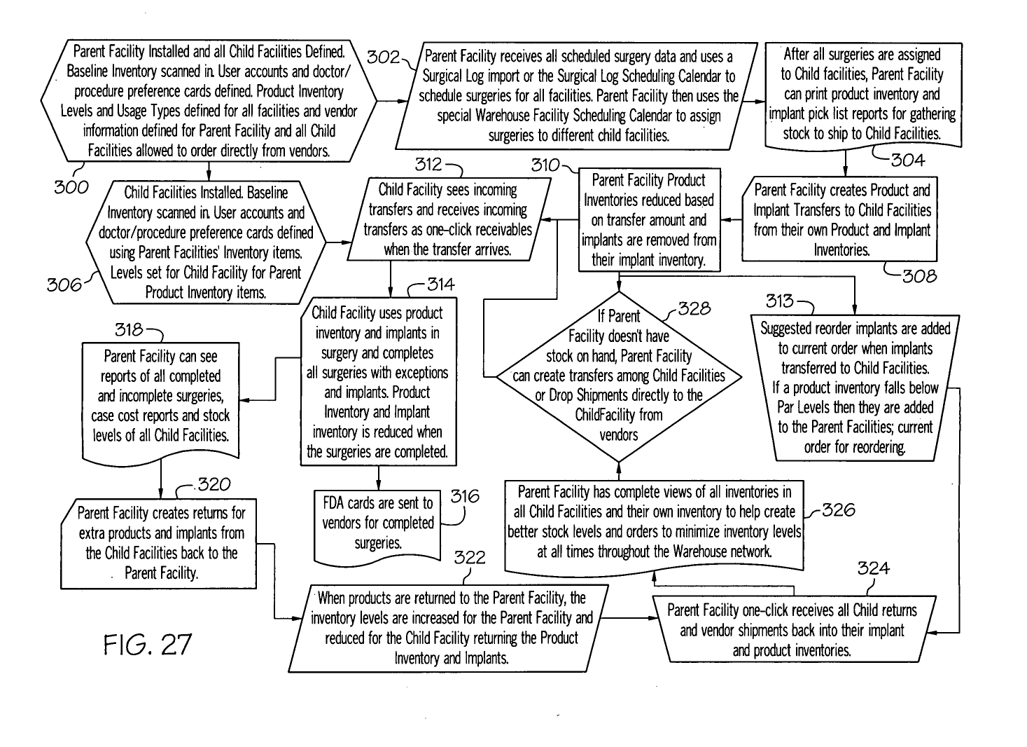

FIG. 27 is a flow chart which further illustrates certain features of the relationship between parent and child facilities. In particular, beginning with step 300, the system and method may be installed at the parent facility and child facilities, and their relationship defined. The baseline inventory (of the parent, or the parent and children) may be entered into the system. Particular user accounts and physician preference cards may be defined. Product inventory levels (i.e., minimum, maximum and par levels) and accounting tracking designations, usage type indicators and the like may be defined for all the facilities (i.e., parent and children). Supplier information may be entered, as well as specifying which party (i.e., parent or child or both) has the ability to order directly from a supplier, and in which circumstances. Next, at step 302, the parent facility receives the scheduled surgery data and imports surgical log data and scheduling information such that, for example, all of the procedures and schedules for all the children facilities may be maintained, controlled or monitored by the parent. At step 304, each of the procedures/surgeries is assigned to a particular child facility, either by the child facility, or the parent facility, or by some cooperative agreement. At this point, the parent facility can identify product inventory and implant pick list reports to identify which products need to be shipped to the child facility.

At step 306, information regarding the child facilities is set up. In particular, baseline inventory for each child is scanned in. User accounts and preference cards specific to each physician may be created or entered relating to inventory items stored at the parent facilities. Inventory levels for the children facilities (i.e., minimum, maximum and par levels) may be defined.

At step 308, the parent facility creates an order transferring items from the parent's inventory to the child facility, i.e., if inventory levels for the child fall too low, or are projected to fall too low. At step 310, the parent facility inventory is correspondingly reduced, and, at step 312, the child facility inventory is increased (such as by using one-click receipt of the receivables). At step 313, after a item has been transferred to the child facility, the system and method may suggest reorder, if appropriate.

At step 314, the child facility uses its item inventory and completes its procedures/surgeries while noting any exceptions. Inventory is reduced upon completion of the surgery/event and, at step 316, FDA cards may be sent to suppliers, analogous to step 272 of FIG. 26.

At step 318, the parent facility has visibility of all the scheduled procedures/surgeries of its child facilities, including completed and uncompleted procedures/surgeries, case cost reports, and stock levels. This allows the parent facility to track existing and predicted levels of inventory on an enterprise-wide level. In addition, as noted at step 320, if it is determined that a child facility is carrying extra items, and/or the items are more urgently needed at another child facility, the parent facility may request the child facility return those items to the parent or directly send the item to another sibling facility. When the items are returned to the parent facility (at step 322), the inventories are increased for the parent facility and reduced for that child facility. At step 324, the shipped items are received by the parent facility, such as by using one-click receiving, thereby receiving the items into inventory. In this manner, as noted at step 326, the parent facility has complete visibility of its own inventory, as well as that of all child facilities, in order to maintain optimum stock levels and minimize required inventory throughout the network (i.e., including child and parent facilities). Finally, at step 328, it is noted that if the parent facility does not have sufficient stock on hand, the parent facility can create transfers among child facilities or drop shipments directly at a child facility from a supplier.

Thus, it can be seen that the system and method disclosed herein provide a complete, integrated system in which inventory levels, cost tracking, item ordering, scheduling and tracking of procedures, case costing, reporting, returns, and other functions are all integrated into a single system and method. Each of the functions described above may be provided or contained in its own module, which can be a block of software, code, instructions or the like which, when run on a computer, provide the desired functions. The modules in the system may be functionally and/or physically separated, but can share data, outputs, inputs, or the like to operate as a single system and provide the functions described herein.

Thus, for example, the system may include an inventory module for tracking the levels of inventory of items, a facility planning module for reserving the use of facilities for procedures, a predictive usage module for predicting the usage of inventoried items for scheduled procedures, an ordering module for receiving and processing orders for items as placed by a user, a predictive costing module that determines the total costs of items which are predicted, by the predictive usage module, to be used, a procedure tracking module for tracking the actual usage of items in a procedure, an actual costing module which calculates the total costs of items actually used in a procedure, etc. Moreover, various different (but, in some cases, related) functions may be combined into a single module, other modules may be provided and/or various other functions not specifically mentioned herein may comprise their own module.

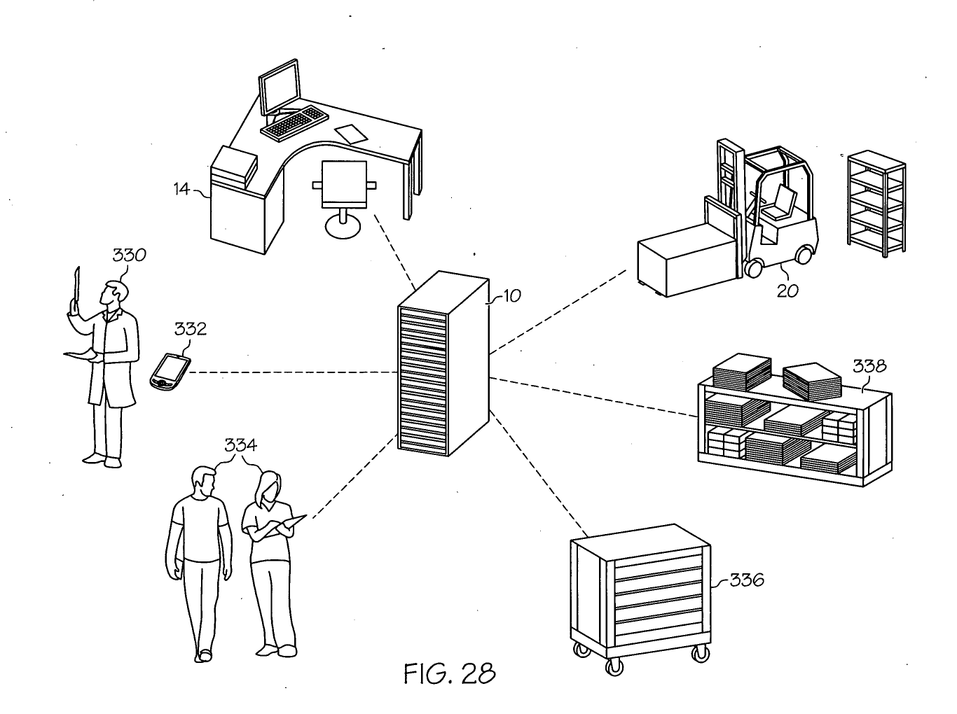

The integrated system and method disclosed herein tracks various forms of data and are useful to a variety of differing end-users, such as inventory managers, physicians, staff, business executives, medical data personnel, owners, directors, regulatory personnel, etc. By way of example, consider a user/physician 330 (FIG. 28) who is an employee of, or has a contract with, a user 14. This physician 330 may be traveling out of town and discover that he/she will be able to return to work a day ahead of schedule. The physician may then remotely log in to the system via his/her mobile phone 332 and check the schedule of the surgical facilities (as can be seen in the screen shot of FIG. 21, for example). Upon finding an appropriate available operating room, the physician 330 may then move the date for one of his/her scheduled procedures up by one day.

As a result of this simple action of advancing the date of the surgical procedure, multiple consequences may follow. For example, the assistants 334 (i.e. nurses, aides, anesthesiologists, etc.) and the patient associated with that procedure may receive notification and automatic calendaring of the change in date of the procedure. A new preference card, which lists all of the supplies needed for the procedure (an example of which is shown in FIG. 16, and which may ultimately may be utilized to generate a cart 336 of associated medical items, as shown in FIG. 28), may be generated.

Adjusting the date of the procedure may cause inventory levels for a particular item, say, gloves, to drop below minimum levels (i.e. in the case, for example, that a shipment of gloves was expected to be received the morning of the originally-scheduled date). In this scenario, the user 14 may receive notification that inventory for gloves has dropped, or will drop, below minimum level. Automatic ordering, or an automatic link to an ordering form such that supplies can be replenished, may be provided to the user 14, and a supplier 20 may pull a box of gloves for shipment to the user 14. The user's associated accounting software may also be notified that an expenditure for gloves may be needed thereby allowing integrated cash flow management and reconciliation of expenses.

The change in procedure date can cause a new projected case costing report (an example of which is shown in FIG. 17) to be generated in place of the original projected case costing report, and may effect other reports 338. Moving the procedure up one day may change the total costs associated with that procedure since differing items (with different costs associated therewith under either the LIFO or FIFO accounting standards) may be utilized in the moved-up procedure. The change in costing may be transmitted to the user's accounting system and effect the user's accounting statements. In addition, moving the day of the procedure adjusts the average storage time of inventory items, and thus may affect the recommended minimum, maximum and par levels. Various other effects can ripple throughout the system having relatively large or small effects.

Thus, it can be seen that a simple data change (moving the date of a procedure) can cause adjustments and accommodations that rapidly and automatically radiate throughout the system. The system and method thus allow efficient and automated changes to reflect such changes, to provide automated adjustments to all data and events tracked by the system and method.

Having described the invention in detail and by reference to the various embodiments, it should be understood that modifications and variations thereof are possible without departing from the scope of the invention.

To the extent that the term “or” is employed in the claims (e.g., A or B) it is intended to mean “A or B or both.” When it is intended to indicate “only A or B but not both”, then the term “A or B but not both” will be utilized. Thus, use of the term “or” in the claims is in an inclusive, and not exclusive, manner.

What is claimed is:

1. A medical item tracking method comprising:
storing inventory levels of items which are for use in medical procedures;
receiving input for reserving the use of a facility for a medical procedure, which medical procedure is projected to use at least one of said inventoried items, and wherein said input identifies at least one individual projected to participate in said medical procedure;
retrieving data relating to the predicted usage of said inventoried item in said medical procedure, wherein said data is based upon the participation of said at least one individual; and
adjusting the levels of inventory based upon the retrieved predicted usage data.

2. The method of claim 1 wherein input received includes a facility identifier, the type of medical procedure, and projected start and end times of the medical procedure.

3. The method of claim 1 further comprising the step of, in response to input received during the receiving step, associating a facility with a particular patient.

4. The method of claim 1 further including the step of receiving information from a user in the form of a request from said user to order items from a supplier, automatically configuring said received information in a format acceptable to or chosen by said supplier, and forwarding said configured information to said supplier.

5. The method of claim 4 further comprising the step of storing information relating to the desired form of orders for a plurality of suppliers in a supplier format database, and wherein the method includes accessing said supplier format database after said information in the form of a request is received from said user to determine the format of information that is acceptable to or chosen by said supplier.

6. The method of claim 4 further including the step of storing at least part of the information forwarded to the supplier, and, upon receipt of at least part of the order from the supplier by the user, retrieving said stored information relating to the order to aid the user in processing the receipt of at least part of the order.

7. The method of claim 1 further including the step of automatically ordering, or suggesting orders to a user, of items if the levels of inventory for said item fall below predefined limits.

8. The method of claim 7 further comprising the step of tracking usage trends of items by said user of items, and wherein the quantity of items in said order or said suggested order is based upon usage trends by said user for the particular item to be ordered.

9. The method of claim 1 further including the step of tracking maximum, minimum, and par levels for each inventoried item, providing an output when the level of a particular inventoried items falls or is projected to fall below the minimum or par levels, and providing an output when the level of a particular inventoried items exceeds or is projected to exceed the maximum level.

10. The method of claim 1 further including the step of tracking maximum, minimum, and par levels for each inventoried item, and automatically adjusting, or automatically providing a suggestion to adjust, the maximum, minimum or par levels for an inventoried item, wherein said adjustment or suggested adjustment is based at least in part upon historical information relating to levels of actual inventory relative to the associated maximum, minimum or par value.

11. The method of claim 1 wherein if said level of inventory in said adjusting step lowers inventory for an item below a predefined level, suggested levels of orders of said item are automatically provided.

12. The method of claim 1 further including the step of providing a reorder form to a user to aid the user in placing an order for items, and wherein at least some fields of the reorder form are automatically populated.

13. The method of claim 1 further including the steps of storing a bundling relationship for a plurality of said items, said bundling relationship defining a ratio of quantities between said plurality of items in a bundle, and effectively displaying said bundling relationship to a user when said user is placing an order for items.

14. The method of claim 12 wherein said bundling relationship is defined based on the projected use of said bundled items in a medical procedure.

15. The method of claim 12 wherein said bundling relationship is based on a pricing model provided by a supplier of said bundled items.

16. The method of claim 1 further comprising the steps of authenticating the identify of an individual, comparing said authenticated identity to an authorization table to determine whether that authenticated individual is authorized, and providing access to the authorized individual to adjust the levels of inventory, reserve the use of a facility for medical procedures or place orders for items.

17. The method of claim 1 further comprising the step of determining the average delivery time for items, and displaying the average delivery time to a user when the user is ordering items.

18. The method of claim 1 further comprising the step of displaying order information to a user, the order information displaying a plurality of suppliers arranged based upon a legal or contractual relationship between said user and that supplier.

19. The method of claim 1 further comprising the step of receiving accounting tracking designations relating to said inventoried items as assigned by a user, and wherein said user-assigned accounting tracking designations are used in accounting for inventory levels for the associated items.

20. The method of claim 1 further comprising the step of receiving usage tracking designations relating to said inventoried items as assigned by a user, wherein said user-assigned usage tracking designations relate to the usage of items by either whole numbers, or percentages, or fractions of whole numbers, and wherein said user-assigned usage tracking designations are used in tracking inventory for the associated items.

21. The method of claim 1 further comprising the step of receiving label information relating to said inventoried items as assigned by said user and displaying said user-defined label information in association with the associated items.

22. The method of claim 1 further including the step of receiving scanned bar code data or RFID tag data from a user, automatically extracting relevant information from said scanned bar code data or RFID tag data to identify an item, and transmitting information relating to said identified item to said user.

23. The method claim 1 wherein said items include uniquely-identified items, and wherein the storing and adjusting steps track unique identifiers associated with each uniquely-identified item.

24. The method of claim 23 wherein said unique identifier associated with each said uniquely-identified items is affixed to packaging that is associated with but separable from said uniquely-identified items.

25. The method of claim 1 wherein the storing step includes storing information related to expiration date, acquisition date, acquisition cost, a physical property, and manufacturer information for said inventoried items.

26. The method of claim 1 wherein the predicted use data is based at least in part upon historical information relating to usage of items in said medical procedure in which said at least one individual participated.

27. The method of claim 1 further comprising the step of calculating the total costs of items which are predicted to be used in said medical procedure.

28. The method of claim 27 wherein the said storing step stores said levels in an inventory database, and wherein said inventory database associates each inventoried item with a cost, and wherein the cost for each item which is predicted to be used in said medical procedure are provided from said inventory database.

29. The method of claim 27 further comprising the step of calculating the total costs of items which were actually used in said medical procedure, and comparing the total costs to the predicted costs.

30. The method of claim 1 further comprising the step of calculating the total costs of items which were actually used in said medical procedure.

31. The method of claim 30 wherein the said storing step stores said levels in an inventory database, and wherein said inventory database associates each inventoried item with a cost, and wherein the cost for each item which was used in said medical procedure are provided from said inventory database.

32. The method of claim 1 further including the steps of tracking the use of inventoried items during said scheduled procedure and adjusting the levels of inventory based upon the tracked usage of said items.

33. The method of claim 1 further comprising the step of receiving scanned bar code data or RFID tag data relating to the usage of items in said medical procedure, and extracting relevant information relating to the identity of said items.

34. The method of claim 1 further comprising the step of providing a user interface such that a user can view the levels of inventory stored in said storing step.

35. The method of claim 33 wherein said user interface is accessible by a user via the internet or via a mobile electronic device.

36. The method of claim 35 further comprising the step of receiving an order for an item from a remotely located user, and automatically forwarding said order to a remotely located supplier.

37. The method of claim 1 further comprising the step of providing an output relating to the availability status of said facility for medical procedures over a particular period of time to aid a user who is seeking to reserve the use of a facility.

38. The method of claim 1 further comprising the step of, in response to said input received in said receiving step, adjusting the availability status of said facility.

39. The method of claim 1 further comprising the step of receiving input adjusting the predicted usage of said inventoried items for said medical procedure.

40. The method of claim 1 wherein said storing, receiving, retrieving, and adjusting steps are each carried out on a computer.

41. The method of claim 1 further including the step of graphically displaying a calendar and said input in said receiving step is provided by a user manipulating said calendar.

42. The method of claim 1 further including the step of automatically sending a bill and replace notification to a supplier based upon the predicted usage data or actual usage data.

43. A medical tracking system comprising:
an inventory module for tracking the levels of inventory of items which are for use in medical procedures;
a facility planning module for reserving the use of a facility for a scheduled medical procedure using at least some of said inventoried items; and
a predictive usage module for predicting the usage of at least some of said inventoried items in said scheduled procedure based upon at least one individual participating in said scheduled procedure, and wherein said predictive usage module is operatively coupled to said facility planning module such that said predicted usage is generated in response to a reservation made through said facility planning module, and wherein said inventory module is operatively coupled to said predictive usage module such information related to said predicted usage of said inventoried items is provided to said inventory module.

44. The system of claim 43 further including an ordering module for receiving and processing orders for items as placed by a user, wherein said ordering module is operatively coupled to said inventory module such that current levels of inventory are displayable to the user when ordering items via said ordering module, and such that information related to placed orders are providable to said inventory module.

45. The system of claim 44 wherein said ordering module or said inventory module is configured to automatically order, or suggest orders, of items, based upon information relating to the levels of inventory tracked by said inventory module.

46. The system of claim 45 wherein said ordering module or inventory module is configured to automatically order, or suggest orders, in quantities based upon usage trends for the particular item to be ordered.

47. The system of claim 44 wherein said ordering module or inventory module is configured to provide a reorder form to a user to aid the user in placing an order for items, and wherein at least some fields of the reorder form are automatically populated based upon information relating to the levels of inventory tracked by said inventory module.

48. The system of claim 43 wherein said inventory module stores a bundle relationship between a plurality of items, said bundle relationship defining a ratio between said items in said bundle, whereby said bundle relationship is defined based on the use of said plurality of items for a specific procedure or a pricing model provided by a supplier of said bundled items.

49. The system of claim 48 wherein said plurality of bundled items have associated or complementary functions.

50. The system of claim 48 wherein said ordering module is configured to identify a needed item from said inventory module which needs to be replenished and form an order or suggested order comprising bundled items which include said needed item.

51. The system of claim 43 further including means for authenticating an individual and comparing said authenticated individual to an authorization table to determine whether that individual is authorized, and wherein said authenticating and comparing means restricts access to said inventory module, said facility planning module or said predictive usage module to authorized individuals.

52. The system of claim 44 wherein said ordering module is configured to receive, from the received information in a format acceptable to or chosen by a supplier identified by said user, and forward the formatted information to said supplier.

53. The system of claim 44 wherein said ordering module stores information relating to a placed order such that when all or some items in said placed order are received by a user, the stored information related to the placed order is a retrievable aid in documenting receipt of said all or some items.

54. The system of claim 44 wherein said ordering module is configured to determine the average delivery time for items ordered via said ordering module.

55. The system of claim 44 wherein said ordering module is configured to display a plurality of suppliers to a user, wherein each supplier is able to supply items to said user, and wherein said suppliers are arranged in said display based upon a legal or contractual relationship between said user and that supplier.

56. The system of claim 43 further including a receiving module which is configured to process received items and provide information regarding said received items to said inventory module, and wherein said receiving module is configured to enable a user to assign an accounting tracking designation to said received items.

57. The system of claim 43 further including a receiving module which is configured to process received items and provide information regarding said received items to said inventory module, and wherein said receiving module is configured to enable a user to assign a label to said received items.

58. The system of claim 43 further including a receiving module which is configured to process received items and provide information regarding said received items to said inventory module, and wherein said receiving module is configured to receive data in the form of a scanned bar code or RFID tag relating to said received items such that said receiving module can automatically extract relevant information from said received data.

59. The system of claim 43 wherein said items tracked by said inventory module include uniquely-identified items and wherein the inventory module tracks a unique identifier associated with each uniquely-identified item.

60. The system of claim 59 wherein said unique identifier associated with each said uniquely-identified items is affixed to packaging that is associated with but separable from said uniquely-identified items.

61. The system of claim 43 wherein said inventory module is configured to track information relating to the usage of items by either whole numbers, or percentages, or fractions of whole numbers.

62. The system of claim 43 wherein said inventory module is configured to track maximum, minimum, and par levels for each inventoried item, and wherein the system is configured to provide an output when the count of a particular inventoried items falls or is projected to fall below the minimum or par levels, and wherein the system is configured to provide an output when the count of a particular inventoried items exceeds or is projected to exceed the maximum level.

63. The system of claim 43 wherein said inventory module is configured to track maximum, minimum, and par levels for each inventoried item, and wherein the system is configured to automatically adjust, or automatically provide a suggestion to adjust, the maximum, minimum or par levels for each inventoried item, wherein said adjustment or suggested adjustment is based at least in part upon historical information relating to levels of actual inventory relative to the associated maximum, minimum or par value.

64. The system of claim 43 wherein the facility planning module includes or is operatively connected to database storing information relating to the associated medical procedure, including the identity of individual participating in the medical procedure, a room identifier, the type of medical procedure, and projected start and end times of the medical procedure.

65. The system of claim 43 wherein said inventory module tracks information related to each inventoried item, including expiration date, acquisition date, acquisition cost, data relating to a physical property or characteristic of the item, and manufacturer information.

66. The system of claim 43 wherein said facility planning module associates a reserved facility with a particular patient.

67. The system of claim 43 wherein said predicted usage by said predictive usage module is based at least in part upon historical information relating to usage of items in the scheduled medical procedure.

68. The system of claim 43 further comprising a predictive costing module operatively coupled to said predictive usage module, wherein said predictive costing module calculates the total costs of items which are predicted, by said predictive usage module, to be used in said scheduled procedures.

69. The system of claim 68 wherein said predictive costing module is operatively coupled to said inventory module such that said predictive costing module assigns costs to the items which are predicted to be used, and wherein said costs are accessed from said inventory module.

70. The system of claim 43 further comprising an actual costing module operatively coupled to said inventory module, wherein said actual costing module calculates the total costs of items actually used in a procedure.

71. The system of claim 70 wherein said actual costing module is operatively coupled to said inventory module such that said actual costing module assigns costs to the items which are used, and wherein said costs are accessed from said inventory module.

72. The system of claim 70 further including a predictive costing module operatively coupled to said predictive usage module, wherein said predictive costing module calculates the total costs of items which are predicted, by said predictive usage module, to be used in said scheduled procedures, and wherein the system is configured to calculate the difference between the total predicted costs of items provided by said predictive costing module and the total costs of items actually used as provided by said actual costing module.

73. The system of claim 43 further including a procedure tracking module operatively coupled to said inventory module, wherein said procedure tracking module is configured to track the occurrence of certain events during a scheduled procedure.

74. The system of claim 73 wherein said procedure tracking module is operatively coupled to said inventory module such that information relating to the actual usage of items is provided to said inventory module.

75. The system of claim 73 wherein said procedure tracking module is configured to receive data in the form of a scanned bar code or RFID tag relating to the usage of items such that said procedure tracking module can extract relevant information relating to events in said scheduled procedure from said received data.

76. The system of claim 43 further comprising a user interface operatively coupled to said inventory module such the levels of inventory of items tracked in said inventory module are viewable by a user.

77. The system of claim 43 wherein said inventory module, said facility planning module and said predictive usage module reside on a central computer and are accessible by a user interface via the internet.

78. The system of claim 77 wherein said central computer is operatively coupled to an item supplier such that said user can place an order for items via said central computer which order is then automatically forwarded to said supplier.

79. The system of claim 43 wherein said facility planning module is configured to provide an output relating to the availability status of a facility for medical procedures over a particular period of time.

80. The system of claim 43 wherein the predicted usage of said inventoried items in said scheduled procedures is adjustable by a user.

81. A medical item tracking method comprising:
storing inventory levels of items which are for use in medical procedures, and their associated costs, in an inventory database;
retrieving data relating to the predicted usage of said inventoried items in a medical procedure, wherein said data is based upon the participation of at least one individual in said scheduled procedure; and
calculating the total costs of items which are predicted to be used in said medical procedure based upon costs stored in said inventory database.

82. The method of claim 81 further comprising the step of calculating the total costs of items which were actually used in said scheduled procedure, and comparing the total costs to the predicted costs.

83. The method of claim 82, wherein the total costs of items which were actually used are based upon costs stored in said inventory database.

84. The method of claim 81 further including the steps of receiving input for reserving the use of a facility for a medical procedure, which medical procedure is projected to use at least one of said inventoried items, and wherein said at least one individual is projected to participate in said medical procedure, and adjusting the levels of inventory based upon the retrieved predicted usage data.

85. The method of claim 81 further including the step of receiving information from a user in the form of a request from said user to order items from a supplier, automatically configuring said received information in a format acceptable to or chosen by said supplier, and forwarding said configured information to said supplier.

86. The method of claim 81 further including the steps of reviewing the levels of inventory in said inventory database, and automatically ordering, or suggesting orders to a user, of items if the levels of inventory fall below predefined limits.

87. The method of claim 81 further including the step of tracking maximum, minimum, and par levels for each inventoried item, providing an output when the level of a particular inventoried items falls or is projected to fall below the minimum or par levels, and providing an output when the level of a particular inventoried items exceeds or is projected to exceed the maximum level.

88. The method of claim 81 further including the steps of storing a bundling relationship for a plurality of said items, said bundling relationship defining a ratio of quantities between said plurality of items in a bundle, and displaying said bundling relationship to a user when said user is placing an order for items.

89. The method of claim 81 further comprising the steps of authenticating the identify of an individual, comparing said authenticated identity to an authorization table to determine whether that authenticated individual is authorized, and providing access to the authorized individual to adjust the levels of inventory or place orders for items.

90. The method of claim 81 further comprising the step of receiving accounting tracking designations relating to said inventoried items as assigned by a user, and wherein said user-assigned accounting tracking designations are used in tracking inventory levels for the associated items in said inventory database.

91. The method of claim 81 further comprising the step of receiving usage tracking designations relating to said inventoried items as assigned by a user, wherein said usage tracking designations relate to the usage of items by either whole numbers, or percentages, or fractions of whole numbers, and wherein said user-assigned usage tracking designations are used in tracking inventory for the associated items in said inventory database.

92. The method claim 81 wherein said items include uniquely-identified items and wherein said inventory database tracks unique identifiers associated with each uniquely-identified item.

93. The method of claim 81 wherein said inventory database stores information related to expiration date, acquisition date, acquisition cost, a physical property, and manufacturer information for said inventoried items.

94. The method of claim 81 wherein the predicted usage data is based at least in part upon historical information relating to usage of items in the scheduled medical procedure in which said at least one individual participated.

95. The method of claim 81 further comprising the step of receiving input adjusting the predicted usage of said inventoried items for said scheduled procedure.

96. A medical tracking system comprising:
an inventory module for tracking the levels of inventory of items which are for use in medical procedures;
a predictive usage module for predicting the usage of at least some of said inventoried items in a medical procedure; and
a predictive costing module operatively coupled to said predictive usage module and said inventory module, wherein said predictive costing module calculates the total costs of items which are predicted, by said predictive usage module, to be used in said scheduled procedure based upon the cost of said items to be used as provided by said inventory module.

97. A medical item tracking method comprising:
storing inventory levels of uniquely-identified items and non uniquely-identified items which are for use in medical procedures, and their associated costs and any associated unique identifiers, in an inventory database;
tracking the actual usage of said inventoried items in a medical procedure; and
upon receipt of an input, automatically calculating the total costs of items which were used in said medical procedure based upon costs stored in said inventory database.

98. A medical tracking system comprising:
an inventory module for tracking the levels of inventory of uniquely identified items and non-uniquely identified items which are for use in medical procedures, wherein said inventory module tracks the costs and any associated unique identifiers;
a procedure tracking module for tracking the actual usage of items in a procedure; and
an actual costing module operatively coupled to said procedure tracking module and said inventory module, wherein said actual costing module calculates the total costs of items actually used in said procedure as provided by said procedure tracking module based upon the cost of said used items as provided by said inventory module.

99. A medical item tracking method comprising:
storing inventory levels of items which are for use in medical procedures;
receiving a request from a user to order items from a supplier;
forwarding said request said supplier;
storing information relating to the request forwarded to the supplier; and
upon receipt of at least part of the order from the supplier by the user, retrieving said stored information relating to the order to aid the user in processing the receipt of said at least part of the order.

100. The method of claim 99 further comprising the step of storing information relating to the desired form of orders for a plurality of suppliers in a supplier format database, accessing said supplier format database after said request is received from said user to determine the format of information that is acceptable to or chosen by said supplier, and automatically configuring information received from said user in a format acceptable to or chosen by said supplier.

101. The method of claim 99 wherein said supplier is an external supplier or an internal warehouse.

102. A medical tracking system comprising:
an inventory module for tracking the levels of inventory of items which are for use in medical procedures; and
an ordering module for receiving and processing orders for items as placed by a user, wherein said ordering module is operatively coupled to said inventory module such that current levels of inventory are displayable to the user when ordering items via said ordering module, and such that information related to placed orders are providable to said inventory module, wherein said ordering module stores information relating to a placed order such that when all or some items in said placed order are received by a user, the stored information related to the placed order is retrievable to document to receipt of said all or some items.

103. A medical item tracking method comprising:
storing, in an inventory database, inventory levels of items which are for use in medical procedures;
receiving input for reserving the use of a facility for a medical procedure, which medical procedure is projected to use at least one of said inventoried items;
tracking the actual usage of said inventoried items in a medical procedure;
automatically adjusting the levels of inventory in said inventory database after said tracking step based upon information obtained during said tracking step; and
automatically ordering, or suggesting orders to a user, of items if the levels of inventory fall below predefined limits.

104. A medical tracking system comprising:
an inventory module for tracking the levels of inventory of uniquely-identified items which are for use in medical procedures;
a facility planning module for reserving the use of a facility for a scheduled medical procedure using at least one of said inventoried items;
a procedure tracking module for tracking the actual usage of items in a medical procedure, wherein said procedure tracking module is operatively coupled to said inventory module such that information relating to the actual usage of items is provided to said inventory module; and
an ordering module for receiving and processing orders for items, wherein said ordering module is operatively coupled to said inventory module such that said ordering module automatically orders, or automatically suggests order levels, of items which are determined are needed to be replenished.

105. A medical tracking system comprising:
an inventory module for tracking the levels of inventory of items which are for use in medical procedures;
a facility planning module for reserving the use of a facility for medical procedures using at least one of said inventoried items; and
a predictive usage module for predicting the usage of at least some of said inventoried items in said scheduled procedures, and wherein said predictive usage module is operatively coupled to said facility planning module such that said predicted usage is generated in response to a reservation made through said facility planning module, and wherein said inventory module is operatively coupled to said predictive usage module such information related to said predicted usage of said inventoried items is provided to said inventory module;
a predictive costing module operatively coupled to said predictive usage module, wherein said predictive costing module calculates the total costs of items which are predicted, by said predictive usage module, to be used in said scheduled procedures; and
an actual costing module operatively coupled to said procedure tracking module, wherein said actual costing module calculates the total costs of items actually used in a procedure.

106. An item tracking method comprising:
storing inventory levels of items which are for use in procedures;
receiving input for reserving the use of a facility for a procedure, which procedure is projected to use at least one of said inventoried items, and wherein said input identifies at least one individual projected to participate in said procedure;
retrieving data relating to the predicted usage of said inventoried items in said procedure, wherein said data is based upon the participation of said at least one individual; and
adjusting the levels of inventory based upon the retrieved predicted usage data.

107. A system comprising a personal electronic device having:
an inventory module for tracking the levels of inventory of items which are for use in medical procedures;
an input module for receiving an input identifying at least one of said inventoried items; and
a receiving module configured to stored information relating to a placed order such that when all or some items in said placed order are received, the stored information related to the placed order is a retrievable to aid in documenting receipt of said all or some items, wherein said receiving module is configured to provide information regarding said received items to said inventory module.
